(19) United States
(12) Patent Application Publication
(10) Pub. No.: US 2012/0189577 A1
(43) Pub. Date: Jul. 26, 2012

(54) USE OF IL-12 TO INCREASE SURVIVAL FOLLOWING ACUTE EXPOSURE TO IONIZING RADIATION

(75) Inventor: Lena A. BASILE, Tujunga, CA (US)

(73) Assignee: Neumedicines, Inc.

(21) Appl. No.: 13/353,179

(22) Filed: Jan. 18, 2012

Related U.S. Application Data
(63) Continuation-in-part of application No. 12/430,016, filed on Apr. 24, 2009.
(60) Provisional application No. 61/125,508, filed on Apr. 24, 2008.

(51) Int. Cl. A61K 38/20; A61P 39/00; A61P 7/00; A61P 37/04; A61P 1/00

(52) U.S. Cl. 424/85.2

(57) ABSTRACT

The present invention is directed to methods of increasing survival following exposure to non-therapeutic radiation comprising administration of IL-12.

### CROSS-REFERENCE TO RELATED APPLICATIONS

The present invention is a continuation-in-part of U.S. patent application Ser. No. 12/430,016, filed Apr. 24, 2009, which claims priority to U.S. Provisional Patent Application No. 61/125,508, filed Apr. 24, 2008, the disclosures of which are hereby incorporated by reference in their entirety.

### BACKGROUND OF THE INVENTION

The hematological effects of acute, high dose total body irradiation (TBI) can lead to death without supportive care, such as hematopoietic transplant, transfusions and other supportive care measures. However, such supportive measures cannot be readily administered to a potentially large number of victims of high dose radiation exposure following a nuclear accident or direct acts of terrorism. If such disasters were to occur, military personnel and civilians alike would be left in great jeopardy. One of the deleterious effects of high dose radiation is the induction of a hematopoietic syndrome. To counteract the potentially lethal effects of hematopoietic syndrome, effective remedial drugs that can be quickly distributed shortly after the radiation incident are in great need.

Currently there are no available drugs that are effective in increasing survival and regenerating hematopoiesis. Moreover, in the face of a radiation-related disaster or act of terrorism, the immediate distribution of such effective drugs to military personnel or civilians, if these were to be available, would not be practically possible. It is anticipated that a lag time of at least several hours, and perhaps 24 hours or greater, would be necessary to distribute such drugs to the scene of such a radiation disaster or act of terrorism. Thus, it is critically important that effective drugs that can be used to increase survival and regenerate hematopoiesis exhibit efficacy at protracted time intervals following acute exposure to ionizing radiation.

Several studies performed in the mid 80's suggested that proinflammatory cytokines could confer radioprotection when administered prior to lethal doses of radiation (Neta et al., Lymphokine Res., 5 Suppl 1:S105-10 (1986); Neta et al., J. Immunol., 136(7):2483-5 (Apr. 1, 1986); Schwartz et al., Immunopharmacol Immunotoxicol., 9(2-3):371-89 (1987)). However, it was recognized by several later studies that the use of IL-12 for prophylactic and therapeutic treatments of lethal irradiation suffered from significant drawbacks. One such adverse effect was that IL-12 administered at a high dose of 1000 ng per mouse radiosensitized, rather than radioprotected, the gastrointestinal tract, resulting in lethal gastrointestinal syndrome in irradiated mice (Neta et al., J. Immunol., 153(9):4230-7 (Nov. 1, 1994)). This work led to the conclusion that IL-12 sensitized the intestinal tract at levels necessary for protection of bone marrow cells (Neta R., Environ Health Perspect., 105 Suppl 6:1463-5 (December 1997).

Consistent with these findings, Hixon et al. (Hixon et al., Biol. Blood Marrow Transplant., 8(6):316-25 (2002)) showed that repeated administration of 500 ng IL-12 to BALBc mice who received bone marrow transplants following lethal whole bode irradiation, resulted in acute lethal toxicity within 4 to 6 days. In contrast, Hixon et al. demonstrate that under identical conditions, BALBc mice that did not receive IL-12 administration recovered 100% of the time.

In the event of a nuclear event, whether accidental or malicious, pre-administration of drugs that promote survival is simply not possible. For example, in the event of a nuclear disaster, where large numbers of people and animals may require therapeutic administration, sufficient time will be required for the large-scale distribution of radiation treatments. Methods are needed for treatment that are effective when administered at protracted times following acute exposure to whole body ionizing radiation.

As such, there remains a need in the art for drugs that are effective in increasing survival and regenerating hematopoiesis when administrated at protracted times following acute exposure to whole body ionizing radiation. The present invention satisfies these and other needs by providing methods of treating a subject following an acute exposure to non-therapeutic whole body ionizing radiation via administration of IL-12 at protracted timepoints.

### BRIEF SUMMARY OF THE INVENTION

The present invention relates to the use of Interleukin-12 (IL-12) for increasing survival and promoting hematopoietic recovery following acute exposure to non-therapeutic ionizing radiation. Use of the methods of the present invention will increase the number of survivors from a radiation-related disaster, such as a terrorist attack with a dirty bomb.

In one aspect, the present invention provides methods for increasing survival, and/or preserving bone marrow function, and/or promoting hematopoietic recovery or restoration in a subject following acute exposure to non-therapeutic, acute whole body ionizing radiation, comprising the administration of a therapeutically effective dose of IL-12.

In a related aspect, the present invention provides pharmaceutically acceptable formulations of IL-12 for increasing survival, and/or preserving bone marrow function, and/or promoting hematopoietic recovery or restoration in a subject following acute exposure to non-therapeutic whole body ionizing radiation.

In another aspect, the present invention provides kits useful for increasing survival, and/or preserving bone marrow function, and/or promoting hematopoietic recovery or restoration in a subject following acute exposure to non-therapeutic whole body ionizing radiation, comprising one or more therapeutically effective dose of IL-12.

Further aspects, objects, and advantages of the invention will become apparent upon consideration of the detailed description and figures that follow.

In one aspect, the present invention provides methods for increasing the probability of survival, and/or preserving bone marrow function, and/or promoting hematopoietic recovery or restoration in a subject following acute exposure to non-therapeutic, acute whole body ionizing radiation, comprising the administration of a therapeutically effective dose of IL-12. In particular, the subject's probability of survival is increased due to at least one or two effects selected from the group consisting of: (a) stimulation of innate immunity effects; (b) protection of the gastrointestinal tract; (c) stimulation of hematopoiesis, and (d) stimulation of antioxidant and anti-apoptotic effects. Moreover, administration of IL-12 may result in increasing the subject's levels of interferon-gamma (IFN-γ), cell survival in the gastrointestinal tract, hematopoiesis, and/or release of erythropoietin (EPO). The subject can be any mammal or animal, including but not limited to a human.

The stimulation of antioxidant and anti-apoptotic effects can result from release of erythropoietin (EPO). In addition, the stimulation of innate immunity effects can result from release of interferon-gamma. Moreover, the probability of survival can be further increased by the release of endogenous IL-12.

In another embodiment of the invention, in the method of the invention for increasing the probability of survival comprising administering IL-12 following an acute exposure to non-therapeutic, acute whole body ionizing radiation, the subject can have a decreased probability of infection, a decrease in tissue damage, or a combination thereof, as compared to a subject exposed to the same level of non-therapeutic whole body ionizing radiation and who has not been given a dose of IL-12. Furthermore, as a result of the method of the invention the subject can have a decreased need for a platelet transfusion, a decreased probability of sepsis, a decreased probability of hemorrhage, or any combination thereof, as compared to a subject exposed to the same level of non-therapeutic whole body ionizing radiation and who has not been given a dose of IL-12.

In this particular method of the invention, IL-12 can directly stimulate the innate immunity effects by binding to the IL-12 receptor on natural killer cells, macrophages, dendritic cells, or any combination thereof.

Exemplary dosages of IL-12 for humans are less than about 300 ng/kg or less than about 100 ng/kg, although any IL-12 dosage described herein can be used in the methods of the invention.

The IL-12 can be administered at any suitable time point following the acute exposure, such as described in more detail herein. For example, IL-12 can be administered between a range of about 6 hours to about 120 hours after the acute radiation exposure, between a range of about 6 hours to about 72 hours after the acute radiation exposure, or between a range of about 48 hours and about 120 hours after the acute exposure. In other embodiments of the invention, IL-12 is administered at about 6 hours, about 12 hours or about 24 hours after the acute radiation exposure.

In one embodiment of the invention, the acute exposure to whole body ionizing radiation is the result of a nuclear event.

The acute exposure in the method of the invention, including but not limited to exposure which is the result of a nuclear event, can be at least about 0.5 Gy or less than about 5.0 Gy, although any exposure (Gy) described herein is encompassed by the methods of the invention.

IL-12 can be administered via any pharmaceutically acceptable means, including but not limited to subcutaneously or intramuscularly.

In yet another embodiment of the invention, supportive care can be given to the subject simultaneously or following the administration of IL-12. Such supportive care comprise one or more of the following: (a) administration of one or more antibiotics; (b) administration of one or more hematopoietic growth factors; and (c) administration of a blood transfusion. Any suitable hematopoietic growth factor can be utilized, such as for example, G-CSF, GM-CSF or EPO.

The invention also encompasses a method for resorting hematopoiesis in a subject, the method comprising administering at least one therapeutically effective dose of IL-12 to the subject following an acute exposure to non-therapeutic whole body ionizing radiation, wherein the dose is administered at least 24 hours after radiation exposure and hematopoiesis is restored via activation of the IL-12 receptor on hematopoietic cells in the bone marrow. The hematopoietic cells can comprise, for example, niche cells and stem cells, and the niche cells can comprise osteoblasts. The subject can be any mammal or animal, including but not limited to a human.

In this method of the invention, hematopoiesis can be restored following activation of the IL-12 receptor on megakaryocytes. The megakaryocytes can be immature. In addition, hematopoiesis can be restored following activation of the IL-12 receptor on osteoblastic cells in the bone marrow, megakaryocyte cells in the bone marrow, hematopoietic stem cells in the bone marrow, or a combination thereof.

The IL-12 can be administered at any suitable time point following the acute exposure, such as described in more detail herein. For example, IL-12 can be administered between a range of about 6 hours to about 120 hours after the acute radiation exposure, between a range of about 6 hours to about 72 hours after the acute radiation exposure, or between a range of about 48 hours and about 120 hours after the acute exposure. In other embodiments of the invention, IL-12 is administered at about 6 hours, about 12 hours or about 24 hours after the acute radiation exposure.

Exemplary dosages of IL-12 for humans are less than about 300 ng/kg or less than about 100 ng/kg, although any IL-12 dosage described herein can be used in the methods of the invention.

Both the foregoing general description and the following brief description of the drawings and detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 (A-C) shows that IL-12 facilitates multilineage hematopoietic recovery at various time points of administration and dosages, and even facilitates significant recovery of all blood groups at 3 hours post radiation (9 Gy). The blood recovery of neutrophils (FIG. 1A), red blood cells (FIG. 1B) and platelets (FIG. 1C) are shown in the graph. The normal threshold count level is depicted for the different blood cell groups (dotted horizontal line). The depicted treatment groups are as follows: IL-12 (200 ng) administered 3 hrs after radiation (♦), IL-12 (100 ng) administered 24 hours before radiation (), IL-12 administered 24 hours before and 2 hr after radiation (100 ng pre/50 ng post) (▪), and IL-12 administered 24 hours before and 2 hr after radiation (100 ng pre/100 ng post) (▴). No control mice remained at 16 days post radiation for blood analysis. Bactrim was removed at 48 days.

FIG. 2 shows survival effects of IL-12 when administered at 6 hours after a lethal dose of radiation (LD_{100/10}) and subcutaneous injection. Female mice C57BL/6 (9 weeks) were used for this experiment (5-6 mice per group).

FIG. 3 shows the results of one aspect of the invention as a Kaplan-Meier plot. Group 1 received vehicle (phosphate buffered saline (PBS)) and Group 2 received an initial dose of IL-12 (120 ng; 18 μg/m²) at 6 hours after radiation, followed by one subsequent dose of IL-12 (100 ng; 15 μg/m² at 3 days post radiation). No antibiotic support was administered. The radiation dose was 8 Gy (0.9 Gy/min); P=0.001 via Mantel Chi-square analysis. Mice did not receive antibiotic support.

FIG. 4 shows remarkable survival effects when administered at 6 hours after a lethal dose of radiation (LD_{100/21}) and subcutaneous injection in male mice. C57BL/6 (14 weeks) were used for this experiment (5-6 mice per group). Mice were first exposed to a non-lethal dose of radiation at 7 Gy. At 6 hours after radiation, mice were either treated with PBS (Group 1) or IL-12 (Group 2 (150 ng; 22.5 μg/m²). All mice survived the 7 Gy dose of radiation. On the next round of radiation, all mice received an 8 Gy radiation dose (0.9 Gy/min). On the second round of radiation, Group 3 received PBS and Group 2 again received the same dose of IL-12 (150 ng; 22.5 μg/m²) that was administered in the first round of radiation, followed by a subsequent dose (100 ng; 15 μg/m²) 48 hrs after the initial dose at 6 hr post radiation.

FIG. 5 shows a Kaplan-Meier plot from the second round of radiation. No antibiotic support was administered; P<0.05 via Mantel Chi-square analysis.

FIG. 6 shows survival effects as a result of IL-12 administration at 24 hours after a lethal dose of radiation (LD_{90/27}) and subcutaneous injection. Female mice C57BL/6 (9 weeks) were used for this experiment (7-8 mice per group). Group 1 received vehicle (phosphate buffered saline (PBS)) and Group 2 received an initial dose of IL-12 (120 ng; 18 μg/m²) at 24 hours after radiation, followed by one subsequent dose of IL-12 (100 ng; 15 μg/m² at 3 days post radiation). No antibiotic support was administered. The radiation dose was 8 Gy (0.9 Gy/min); P=0.001 via Mantel Chi-square analysis.

FIG. 7 shows a Kaplan-Meier plot at 27 days in the experimental timeline. No antibiotic support was used. P=0.005 via Mantel Chi-square analysis.

FIG. 8 (A-B) shows the results of female C57BL/6 mice (10 per group), which were irradiated with an LD₈₀ dose of acute irradiation. The mice were then administered the indicated amounts of IL-12 as either (FIG. 8A) a double dose at 24 hours and 72 hours post irradiation, or (FIG. 8B) a single dose at 24 hour post irradiation. The survival rates for each of the groups are shown.

FIG. 9 shows the results of female C57BL/6 mice (10 per group), which were irradiated with an LD₈₀ dose of acute irradiation. The mice were then administered the indicated amounts of IL-12 as either (FIG. 9A) a double dose at 24 hours and 72 hours post irradiation, or (FIG. 9B) a single dose at 24 hour post irradiation. The average weight of the surviving mice are plotted as a function of time in days.

FIG. 10 illustrates a stratified K-M plot for double dose experiments performed with female C57BL/6 mice (10 per group). Briefly, the mice were irradiated with an LD₈₀ dose of acute irradiation followed by administration of a double dose of IL-12, as described in example 6, at 24 and 72 hours post irradiation. Survival analysis did not indicate a significant overall effect of IL-12 dose on survival (p<0.59; Tarone-Ware test).

FIG. 11 illustrates a K-M plot of survival for group 1 (0 ng IL-12, vehicle control; (▴)) and group 7 (300 ng IL-12; 45 μg/m²; ()) female C57BL/6 mice irradiated with an LD₈₀ dose of acute irradiation followed by administration of IL-12 at 24 and 72 hours post irradiation. Survival analysis reveals that administration of 300 ng (45 μg/m²) IL-12 at both 24 and 72 hours post irradiation resulted in a significant increase in survival (p<0.03; Tarone-Ware and Mantel-Cox tests).

FIG. 12 illustrates a stratified K-M plot for single dose experiments performed with female C57BL/6 mice (10 per group). Briefly, the mice were irradiated with an LD₈₀ dose of acute irradiation followed by administration of a single dose of IL-12, as described in example 6, at 24 hours post irradiation. Survival analysis indicates a significant overall effect of IL-12 dose on survival (p<0.02; Tarone-Ware test).

FIG. 13 illustrates a K-M plot of survival for group 1 (0 ng IL-12, vehicle control; (▴)), group 2 (40 ng IL-12; 6 μg/m² (▪)), and group 7 (300 ng IL-12; 45 μg/m² ()) female C57BL/6 mice irradiated with an LD₈₀ dose of acute irradiation followed by administration of IL-12 at 24 hours post irradiation. Survival analysis reveals that administration of 40 ng (6 μg/m²) and 300 ng (45 μg/m²) IL-12 at 24 hours post irradiation resulted in a significant increase in survival (p<0.001; Tarone-Ware test).

FIG. 14 illustrates a dose response curve for single dose administration of IL-12 24 hours after irradiation. Female C57BL/6 mice irradiated with an LD₈₀ dose of acute irradiation followed by administration of IL-12 at 24 hours post irradiation, as in example 6. Survival time and percent weight loss are plotted as a function of administered IL-12 dose.

FIG. 15 illustrates a survival curve for female C57BL/6 mice irradiated with various acute doses of ionizing radiation.

FIG. 16A is a graph of percent survival vs. time for 8 mice administered rMuIL-12 (HemaMax™) at a dose of 10 ng/mouse at 24 hours and at 72 hours after total body irradiation, with 7 control mice given a vehicle only. FIG. 16B is a graph of percent survival vs. time for mice administered rMuIL-12 (HemaMax™) at a dose of 30 ng/mouse at 24 (n=10), 48 (n=10), and 72 (n=10) hours after total body irradiation, with 10 control mice given a vehicle only. FIG. 16C is a graph of percent survival vs. time for mice administered rMuIL-12 (HemaMax™) at doses of either 2 (n=10) or 18 (n=10) ng/mouse about 24 hours after irradiation, with 10 control mice given a vehicle only.

FIG. 17 is a graph of percent survival vs. time for mice administered rMuIL-12 (HemaMax™) at a dose of 20 ng/mouse at about 24 hours after irradiation. Mice received radiation doses of 8.6 Gy (LD_{70/30}) (n=10), 8.8 Gy (LD_{90/30}) (n=10), and 9.0 Gy (LD_{100/30}) (n=10). For each group of mice, a control group was given the same radiation doses with no subsequent rMuIL-12 administration (n=10 for each radiation dosage group)

FIGS. 18A-D are graphs of rMuIL-12 and IFN-γ plasma concentrations over time in mice treated with various doses of rMuIL-12. FIG. 18A is a graph of plasma cytokine concentration (rMuIL-12 and IFN-γ) (pg/mL) vs. time in mice: (1) rMuIL-12 (HemaMax™) at 10 ng/mouse and no radiation; (2) rMuIL-12 (HemaMax™) at 10 ng/mouse with radiation; (3) IFN-γ and no radiation; and (4) IFN-γ and irradiation. FIG. 18B is a graph of plasma cytokine concentration (pg/mL) vs. time in mice: (1) rMuIL-12 (HemaMax™) at 20 ng/mouse and no radiation; (2) rMuIL-12 (HemaMax™) at 20 ng/mouse with radiation; (3) IFN-γ and no radiation; and (4) IFN-γ and irradiation. FIG. 18C is a graph of plasma cytokine concentration (pg/mL) vs. time in mice: (1) rMuIL-12 (HemaMax™) at 40 ng/mouse and no radiation; (2) rMuIL-12 (HemaMax™) at 40 ng/mouse with radiation; (3) IFN-γ and no radiation; and (4) IFN-γ and irradiation. FIG. 18D is a graph of plasma cytokine concentration (pg/mL) vs. time in mice: (1) rMuIL-12 (HemaMax™) at 200 ng/mouse and no radiation; (2) rMuIL-12 (HemaMax™) at 200 ng/mouse with radiation; (3) IFN-γ and no radiation; and (4) IFN-γ and irradiation

FIG. 19 is a bar graph of plasma concentrations of erythropoietin (EPO) (pg/mL) from mice exposed to 0 ng/mouse, 10 ng/mouse, 20 ng/mouse, 40 ng/mouse, or 200 ng/mouse of rMuIL-12 (HemaMax™), both with and without irradiation. Irradiated mice were exposed to 8.6 Gy of TBI resulting in an LD_{100/30}. Mice in the rMuIL-12 group were treated 24 hours following radiation exposure.

FIGS. 20A-G are a series of photomicrographs of mouse femoral bone marrow stained for IL-12Rβ2, as described in Example 8. The mice were subjected to total body irradiation (8 Gy) and received either vehicle or rMuIL-12 (HemaMax™) at various times post-irradiation. FIG. 20A shows photomicrographs of mouse femoral bone marrow stained for IL-12Rβ2 from non-irradiated, untreated mice; the photomicrographs show mature and immature megakaryocytes and myeloid progenitor cells in the metamyelocyte. FIG. 20B shows photomicrographs of bone marrow from mice treated only with vehicle and subjected to an LD_{30/30} of TBI (8.0 Gy). FIG. 20C shows photomicrographs of bone marrow from mice treated with various dosing regimens of rMuIL-12 (irradiation+20 ng/mouse rMuIL-12 at 24 hours following radiation exposure). FIG. 20D shows photomicrographs of bone marrow from mice treated with various dosing regimens of rMuIL-12 (irradiation+20 ng/mouse rMuIL-12 at 2 days and 24 hours following radiation exposure). FIG. 20E shows photomicrographs of bone marrow from mice treated with various dosing regimens of rMuIL-12 (irradiation+20 ng/mouse rMuIL-12 at 3 days and 24 hours following radiation exposure). FIG. 20F shows photomicrographs of bone marrow from mice treated with various dosing regimens of rMuIL-12 (irradiation+20 ng/mouse rMuIL-12 at 4 days and 24 hours following radiation exposure). FIGS. 20C-F show varying levels of hematopoietic reconstitution, which was characterized with the presence of IL-12Rβ2—expressing myeloid progenitors, megakaryocytes, and osteoblasts. FIG. 20G shows photomicrographs of bone marrow from mice treated with various dosing regimens of rMuIL-12 (irradiation+20 ng/mouse rMuIL-12 at 24 hours following radiation exposure), showing that the bone marrow lacked megakaryocytes even with signs of some regeneration.

FIGS. 21A-C are a series of photomicrographs of femoral bone marrow stained for IL-12Rβ2 and Sca-1 (FIG. 21A), or IL-12Rβ2 and osteocalcin (FIG. 21B, as described in Example 12. FIGS. 21A and C show tissue sections obtained 30 days after total body irradiation; FIG. 21B shows tissue sections obtained 12 days after total body irradiation. FIG. 21C shows IL-12Rβ2 and Sca-1 costaining Magnification is 100×.

FIG. 22A is a photomicrograph of mouse jejunal crypts stained for IL-12Rβ2, as described in Example 12. FIG. 22B is a series of photomicrographs of jejunal crypts from mice treated with rMuIL-12 (HemaMax™) (0 ng/mouse, 10 ng/mouse, 20 ng/mouse, 40 ng/mouse, or 200 ng/mouse), either without irradiation (top panels) or with irradiation (8.6 Gy; bottom panels). Magnification is 400×.

FIG. 23 is a plot of plasma AUC_{Last} for rMuIL-12 (HemaMax™) (pg.h/mL) versus dose of rMuIL-12 (HemaMax™) administered to non-irradiated mice and non-irradiated monkeys.

FIGS. 24A-C are graphs of temporal kinetics of rHuIL-12, IFN-γ, IL-18, erythropoietin, and neopterin following administration of rHuIL-12 to monkeys. FIG. 24A shows plasma rHuIL-12 (HemaMax™) concentration (pg/mL) and plasma IFN-γ concentration (pg/mL) vs. time (hours) following rHuIL-12 (HemaMax™) dosages of 250 ng/Kg and 1000 ng/Kg. FIG. 24B shows plasma concentrations (pg/mL) of IL-18 and EPO vs. time following rHuIL-12 (HemaMax™) dosages of 250 ng/Kg and 1000 ng/Kg. FIG. 24C shows plasma IL-15 concentration (pg/mL) and plasma Neopterin concentration (ng/mL) vs. time following rHuIL-12 (HemaMax™) dosages of 250 ng/Kg and 1000 ng/Kg.

FIGS. 25A and B are photomicrographs of human and monkey bone marrow (FIG. 25A) and jejunum/ileum (FIG. 25B) stained for IL-12Rβ2

FIGS. 26A and B are graphs of survival for monkeys exposed to irradiation and given one or two doses of rHuIL-12 at various concentrations. FIG. 26A shows percent survival vs. time for vehicle (n=8), 100 ng/kg rHuIL-12 at 24 hours (n=7), 250 ng/kg rHuIL-12 at 24 hours (n=8), 100 ng/kg rHuIL-12 at 24 hours and day 7 (n=7), and 250 ng/kg rHuIL-12 at 24 hours and day 7 (n=8). FIG. 26B shows percent survival vs. time for vehicle (n=8) and rHuIL-12 group (pooled; n=31).

FIG. 27A-B are graphs of temporal kinetics of leukocyte (FIG. 27A) and platelet (FIG. 27B) counts for monkeys that received two different doses (100 ng/kg and 250 ng/kg of rHuIL-12) of rHuIL-12 at 24 hours following irradiation.

FIGS. 28A-C are graphs of body weight over time for monkeys that received two different doses of rHuIL-12 following irradiation. FIG. 28A shows body weight (kg) vs. time following an rHuIL-12 (HemaMax™) dose of 100 ng/Kg, with vehicle (n=8), rHuIL-12 at 24 hours (n=7), and rHuIL-12 at 24 hours and 1 day (n=8). FIG. 28B shows body weight (kg) vs. time following an rHuIL-12 (HemaMax™) dose of 250 ng/Kg, with vehicle (n=8), rHuIL-12 at 24 hours (n=8), and rHuIL-12 at 24 hours and 1 day (n=8). FIG. 28C shows body weight (kg) vs. time following an rHuIL-12 (HemaMax™) dose of 100 ng/Kg, with vehicle (n=8), rHuIL-12 at 24 hours (n=7), and rHuIL-12 at 24 hours and 1 day (n=8).

FIG. 29 is a schematic summarizing the multiple effects of recombinant IL-12 on various types of cells following radiation exposure. ↑=increase; ↓=decrease; HSC=Hematopoietic stem cells; NK cells=natural killer cells.

### DETAILED DESCRIPTION

### I. Definitions

“Interleukin-12 (IL-12)” as used herein includes any recombinant IL-12 molecule that yields at least one of the properties disclosed herein, including native IL-12 molecules, variant IL-12 molecules and covalently modified IL-12 molecules, now known or to be developed in the future, produced in any manner known in the art now or to be developed in the future. Generally, the amino acid sequence of the IL-12 molecule used in embodiments of the invention is the canonical human sequence related to IL-12p70. IL-12 comprises two subunits, IL-12A (p35) and IL-12 B (P40) Polymorphisms, however, are known to exist for IL-12, especially in the p35 subunit. In particular, a known polymorphism can exist at amino acid 247 of the p35 human subunit, where methionine is replaced by threonine. Still other embodiments of the invention include IL-12 molecules where the native amino acid sequence of IL-12 is altered from the native sequence, but the IL-12 molecule functions to yield the properties of IL-12 that are disclosed herein. Alterations from the native, species specific amino acid sequence of IL-12 include changes in the primary sequence of IL-12 and encompass deletions and additions to the primary amino acid sequence to yield variant IL-12 molecules. An example of a highly derivatized IL-12 molecule is the redesigned IL-12 molecule produced by Maxygen, Inc. (Leong et al., Proc Natl Acad Sci USA., 100(3):1163-8 (Feb. 4, 2003)), where the variant IL-12 molecule is produced by a DNA shuffling method. Also included are modified IL-12 molecules included in the methods of invention, such as covalent modifications to the IL-12 molecule that increase its shelf life, half-life, potency, solubility, delivery, etc., additions of polyethylene glycol groups, polypropylene glycol, etc., in the manner set forth in U.S. Pat. No. 4,640,835; 4,496,689; 4,301,144; 4,670,417; 4,791,192 or 4,179,337, each of which is hereby incorporated by reference. One type of covalent modification of the IL-12 molecule is introduced into the molecule by reacting targeted amino acid residues of the IL-12 polypeptide with an organic derivatizing agent that is capable of reacting with selected side chains or the N- or C-terminal residues of the IL-12 polypeptide. Other IL-12 variants included in the present invention are those where the canonical sequence has been altered to increase the glycosylation pattern of the resultant IL-12 molecule, as compared with the native, non-altered IL-12. This method has been used to generate second generation molecules of erythropoietin, referred to as Aranesp. Both native sequence IL-12 and amino acid sequence variants of IL-12 may be covalently modified. Also as referred to herein, the IL-12 molecule can be produced by various methods known in the art, including recombinant methods. Since it is often difficult to predict in advance the characteristics of a variant IL-12 polypeptide, it will be appreciated that some screening of the recovered variant will be needed to select the optimal variant. A preferred method of assessing a change in the properties of variant IL-12 molecules is via the lethal irradiation rescue protocol disclosed below. Other potential modifications of protein or polypeptide properties such as redox or thermal stability, hydrophobicity, susceptibility to proteolytic degradation, or the tendency to aggregate with carriers or into multimers are assayed by methods well known in the art.

“Acute Radiation Syndrome” in humans as used herein includes an acute radiation exposure of 2 Gy or greater.

Hematopoietic Syndrome” as used herein includes damage to the bone marrow compartment which results in pancytopenia, i.e., a deficiency in peripheral blood cell counts for all blood cell types, namely white blood cells, red blood cells and platelets. Hematopoietic Syndrome also refers to loss of hematopoietic progenitor and stem cells in the bone marrow compartment.

“Survival” as used herein includes an increase in survival that can be measured in non-human species as compared to control groups, such as mice or non-human primates.

“Hematopoietic Recovery” as used herein includes early recovery of peripheral blood cell counts for white blood cells, red blood cells and platelets, as compared to control groups and as measured in non-human species, such as mice or non-human primates.

“Preservation of bone marrow function” as used herein includes early recovery of cellularity or colony forming units in the bone marrow compartment, or any other measure of bone marrow function, as compared to control groups and as measured in non-human species, such as mice and non-human primates.

A “therapeutically effective amount or dose” or “sufficient amount or dose” as used herein includes a dose that produces effects for which it is administered, for example, a dose sufficient for increasing survival, and/or preserving bone marrow function, and/or promoting hematopoietic recovery or restoration in a subject that has been exposed to an acute dose of whole body ionizing radiation. The exact dose will depend on the purpose of the treatment and the timing of the IL-12 administration, certain characteristics of the subject to be treated, the total amount or timing of irradiation, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Generally, a dose of a therapeutic agent, according to the methods and compositions of the present invention, can be expressed in terms of the total amount of drug to be administered (i.e., ng, μg, or mg). Preferably, the dose can be expressed as a ratio of drug to be administered to weight or surface area of subject receiving the administration (i.e., ng/kg, μg/kg, ng/m², or mg/m²). When referring to a dose in terms of the mass to be administered per mass of subject (i.e., ng/kg), it will be understood that doses are not equivalent between different animals, and thus conversion factors will need to be used to ensure that one animal receives the same dose equivalent as another animal. Suitable factors for the conversion of a mouse “dose equivalent” to a “dose equivalent” of a different animal are given in the look-up table below. Thus, in a preferred embodiment, doses are given in terms of mass to surface area (i.e., ng/m²), which are equivalent for all animals. The following basic conversion factors can be used to convert ng/kg to ng/m²: mouse=3.0, hamster=4.1, rat=6.0, guinea pig=7.7, human=38.0 (Cancer Chemother. Repts., 50(4):219 (1966)).

TABLE 1
Conversion factors and equivalent doses for several animals.

| | Weight | Total Dose | Dose | Dose | Conversion |
|---|---|---|---|---|---|
| Species | (kg) | (ng) | (ng/kg) | (ng/m²) | Factor |
| Human | 65 | 25655.82 | 394.7 | 15000 | 0.0794 |
| Mouse | 0.02 | 99.47 | 4973.44 | 15000 | 1.0000 |
| Hamster | 0.03 | 130.2 | 4339.87 | 15000 | 0.8726 |
| Rat | 0.15 | 381.12 | 2540.8 | 15000 | 0.5109 |
| Guinea Pig | 1.00 | 1335 | 1335 | 15000 | 0.2684 |
| Rabbit | 2 | 2381.1 | 1190.55 | 15000 | 0.2394 |
| Cat | 2.5 | 2956.44 | 1182.57 | 15000 | 0.2378 |
| Monkey | 3 | 3681.75 | 1227.25 | 15000 | 0.2468 |
| Dog | 8 | 6720 | 840 | 15000 | 0.1689 |

As used herein, the term “intermediate dose” includes doses between a range of about 15 μg/m² and about 75 μg/m², or between a range of about 20 μg/m² and about 75 μg/m², between a range of about 22.5 μg/m² and about 67.5 μg/m², between a range of about 22.5 μg/m² and about 52.5 μg/m², between a range of about 30 μg/m² and about 60 μg/m², between a range of about 37.5 μg/m² and about 52.5 μg/m², and all doses and ranges in-between.

As used herein, the term “low dose” includes doses less than about or about 15 μg/m², less than about or about 14 μg/m², less than about or about 13 μg/m², less than about or about 12 μg/m², less than about or about 11 μg/m², less than about or about 10 μg/m², less than about or about 9 μg/m², less than about or about 8 μg/m², less than about or about 7 μg/m², less than about or about 6 μg/m², less than about or about 5 μg/m², less than about or about 4 μg/m², less than about or about 3 μg/m², less than about or about 2 μg/m², less than about or about 1 μg/m², less than about or about 900 ng/m², less than about or about 800 ng/m², less than about or about 700 ng/m², less than about or about 600 ng/m², less than about or about 500 ng/m², less than about or about 400 ng/m², less than about or about 300 ng/m², less than about or about 200 ng/m², or less than about or about 100 ng/m². In certain embodiments, a low dose includes an ultralow dose.

As used herein, the term “ultralow dose” includes doses less than about or about 3 μg/m², less than about or about 2 μg/m², less than about or about 1 μg/m², less than about or about 900 ng/m², less than about or about 800 ng/m², less than about or about 700 ng/m², less than about or about 600 ng/m², less than about or about 500 ng/m², less than about or about 400 ng/m², less than about or about 300 ng/m², less than about or about 200 ng/m², or less than about or about 100 ng/m².

### II. Embodiments

In one aspect, the present invention is based on the surprising discovery that Interleukin-12 (IL-12) increases the survival of subjects exposed to lethal and sub-lethal acute doses of non-therapeutic whole body ionizing radiation. Significantly, it was found that administration of IL-12 following acute doses of ionizing radiation resulted in the regeneration of hematopoietic activity and peripheral blood cell counts in lethally irradiated mice, yielding complete hematopoietic recovery without the use of transplanted cells.

In another aspect, the present invention provides methods for increasing the probability of survival, and/or preserving bone marrow function, and/or promoting hematopoietic recovery or restoration in a subject following acute exposure to non-therapeutic, acute whole body ionizing radiation, comprising the administration of a therapeutically effective dose of IL-12. In particular, the subject's probability of survival is increased due to at least one or two effects selected from the group consisting of: (a) stimulation of innate immunity effects; (b) and protection of the gastrointestinal tract; (c) stimulation of hematopoiesis, and (d) stimulation of antioxidant and anti-apoptotic effects. Moreover, administration of IL-12 may result in increasing the subject's levels of interferon-gamma (IFN-γ), cell survival in the gastrointestinal tract, hematopoiesis, and/or release of erythropoietin (EPO). The subject can be any mammal or animal, including but not limited to a human.

The stimulation of antioxidant and anti-apoptotic effects can result from release of erythropoietin (EPO). In addition, the stimulation of innate immunity effects can result from release of interferon-gamma. Moreover, the probability of survival can be further increased by the release of endogenous IL-12.

As a result of the method of the invention, the subject can have a decreased probability of infection, a decrease in tissue damage, or a combination thereof, as compared to a subject exposed to the same level of non-therapeutic whole body ionizing radiation and who has not been given a dose of IL-12. Furthermore, as a result of the method of the invention the subject can have a decreased need for a platelet transfusion, a decreased probability of sepsis, a decreased probability of hemorrhage, or any combination thereof, as compared to a subject exposed to the same level of non-therapeutic whole body ionizing radiation and who has not been given a dose of IL-12.

In this particular method of the invention, IL-12 can directly stimulate the innate immunity effects by binding to the IL-12 receptor on natural killer cells, macrophages, dendritic cells, or any combination thereof.

The invention also encompasses a method for resorting hematopoiesis in a subject, the method comprising administering at least one therapeutically effective dose of IL-12 to the subject following an acute exposure to non-therapeutic whole body ionizing radiation, wherein the dose is administered at least 24 hours after radiation exposure and hematopoiesis is restored via activation of the IL-12 receptor on hematopoietic cells in the bone marrow. The hematopoietic cells can comprise, for example, niche cells and stem cells, and the niche cells can comprise osteoblasts. The subject can be any mammal or animal, including but not limited to a human.

In this method of the invention, hematopoiesis can be restored following activation of the IL-12 receptor on megakaryocytes. The megakaryocytes can be immature. In addition, hematopoiesis can be restored following activation of the IL-12 receptor on osteoblastic cells in the bone marrow, megakaryocyte cells in the bone marrow, hematopoietic stem cells in the bone marrow, or a combination thereof.

### A. Timing of IL-12 Administration

Advantageously, it was found that IL-12 has the unique and remarkable property of being able to confer survival on lethally irradiated mammals when administered at protracted time points after radiation. Specifically, it was found that IL-12 can rescue about 90 to about 100% of mammals when administered at time points such as about 6 hours or about 24 hours after radiation.

Advantageously, as provided by the methods of the present invention, administration of IL-12 may occur during any suitable time period following exposure to acute whole body radiation, up to and including about a week after exposure. Although the total dose of acute radiation will factor into the time period in which IL-12 should be administered, according to one embodiment, IL-12 may be administered at any time up to about 120 hours following exposure to radiation. In other embodiments, IL-12 can be administered at any time up to about 96 hours post-irradiation, up to about 72 hours post-irradiation, or at a time up to about 60 hours, about 48 hours, about 36 hours, about 24 hours, about 18 hours, about 12 hours, about 8 hours, about 6 hours, or less following exposure to radiation.

For example, IL-12 can be administered between a range of about 6 hours to about 120 hours after the acute radiation exposure, between a range of about 6 hours to about 72 hours after the acute radiation exposure, or between a range of about 48 hours and about 120 hours after the acute exposure. In other embodiments of the invention, IL-12 is administered at about 6 hours, about 12 hours or about 24 hours after the acute radiation exposure.

In one specific embodiment, IL-12 is administered to a subject in need thereof between a range of about 1 hour to about 72 hours after exposure to ionizing radiation. In another embodiment, IL-12 is administered between a range of about 1 hour and about 24 hours after exposure, or between a range of about 6 hours and about 24 hours following exposure to an acute dose of whole body ionizing radiation. Of great importance for the usefulness of IL-12 in the face of a radiation disaster is the fact that IL-12 can be administered at protracted time points following acute exposure to ionizing radiation. IL-12 is effective at any time point post radiation up to one week, but can provide especially high effectiveness at time points up to 96 hours post radiation.

In certain other aspects, IL-12 can be administered at any reasonable time point post radiation event, and be effective in increasing survival, and/or preserving bone marrow function, and/or promoting hematopoietic recovery. IL-12 administered at time points of up to 3 hours, 6 hours, 24 hours, 48 hours, and/or 72 hours are efficacious in increasing survival, preserving bone marrow function, and promoting hematopoietic recovery. However, on one aspect, IL-12 acts on a residual subpopulation of hematopoietic stem cells, and thus it is expected to be effective at 96 hours, 120 hours and up to one week following acute exposure to ionizing radiation.

IL-12 can be administered at any time point before or after radiation exposure. In one embodiment of the invention, IL-12 is administered at least about 24 hours or more following exposure to radiation. Other time points for administration following radiation exposure include about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, or about any combination thereof with multiple IL-12 administrations (e.g., at 12 hours and 48 hours.

### B. IL-12 Dosing and Dosages

Generally the IL-12 doses used in the methods for increasing survival in a subject, increasing levels of erythropoietin (EPO) in subject, increasing levels of INF-γ in a subject, and/or preserving bone marrow function, and/or promoting hematopoietic recovery or restoration in a subject following an acute exposure to non-therapeutic whole body ionizing radiation will be high enough to be effective for the treatment of a radiation syndrome, but low enough to mitigate negative side effects associated with IL-12 administrations, including for example, radiosensitivity of the GI tract and INF-γ up-regulation.

In one aspect, a single dose of IL-12 is sufficient to confer significant survival, bone marrow preservation, increase levels of erythropoietin (EPO) in subject, increasing levels of INF-γ in a subject, and promote of hematopoietic recovery. In other aspects, IL-12 may be administered in more than one dose, such as about 2, about 3, about 4, about 5 or more doses.

Accordingly, in one aspect, the present invention provides a method for increasing survival in a subject, increasing levels of erythropoietin (EPO) in subject, increasing levels of INF-γ in a subject, and/or preserving bone marrow function, and/or promoting hematopoietic recovery or restoration comprising the administration of a dose of IL-12 to the subject following an acute exposure to non-therapeutic whole body ionizing radiation. In one embodiment, the dose of IL-12 is less than about 100 μg/m². In one embodiment, the dose of IL-12 is less than about 75 μg/m². In another embodiment, the low dose can be between about 1 μg/m² and about 100 μg/m², such as about 1 μg/m², about 5 μg/m², about 10 μg/m², about 15 μg/m², about 20 μg/m², about 25 μg/m², about 30 μg/m², about 35 μg/m², about 40 μg/m², about 45 μg/m², about 50 μg/m², about 55 μg/m², about 60 μg/m², about 65 μg/m², about 70 μg/m², about 75 μg/m², about 80 μg/m², about 85 μg/m², about 90 μg/m², about 95 μg/m² or about 100 μg/m² and all doses in-between. In a particular embodiment, the preferred dose of IL-12 is less than about 75 μg/m². In one embodiment, the dose of IL-12 is either between a range from about 100 μg/m² to about 18 μg/m² or between a range from about 15 μg/m² to about 1 μg/m².

In another aspect, the present invention provides methods for increasing survival in a subject, increasing levels of erythropoietin (EPO) in subject, increasing levels of INF-γ in a subject, and/or preserving bone marrow function, and/or promoting hematopoietic recovery or restoration comprising the administration of an intermediate dose of IL-12 to the subject following an acute exposure to non-therapeutic whole body ionizing radiation. In one embodiment, the intermediate dose of IL-12 is between a range of about 15 μg/m² and about 100 μg/m², or between a range of about 20 μg/m² and about 75 μg/m², between a range of about 22.5 μg/m² and about 67.5 μg/m², between a range of about 22.5 μg/m² and about 52.5 μg/m², between a range of about 30 μg/m² and about 60 μg/m², between a range of about 37.5 μg/m² and about 52.5 μg/m², and all doses and ranges in-between. In a particular embodiment, the preferred intermediate dose of IL-12 is between a range of about 22.5 μg/m² and about 52.5 μg/m².

In another aspect, the present invention provides methods for increasing survival in a subject, increasing levels of erythropoietin (EPO) in subject, increasing levels of INF-γ in a subject, and/or preserving bone marrow function, and/or promoting hematopoietic recovery or restoration comprising the administration of a low dose of IL-12 to the subject following an acute exposure to non-therapeutic whole body ionizing radiation. In one embodiment, the low dose of IL-12 is less than about 15 μg/m². In another embodiment, the low dose can be between about 3 μg/m² and about 12 μg/m², such as about 3 μg/m², about 4 μg/m², about 5 μg/m², about 6 μg/m², about 7 μg/m², about 8 μg/m², about 9 μg/m², about 10 μg/m², about 11 μg/m², or about 12 μg/m² and all doses in-between. In a particular embodiment, the preferred low dose of IL-12 is about 6 μg/m².

In one particular embodiment, a method is provided for increasing survival, increasing levels of erythropoietin (EPO) in subject, increasing levels of INF-γ in a subject, and/or preserving bone marrow function, and/or promoting hematopoietic recovery or restoration in a human comprising the administration of a low dose of IL-12 to the human following an acute exposure to non-therapeutic whole body ionizing radiation. In a specific embodiment, the method for treating a human comprises administering a low dose of IL-12 in a range between about 1 hour and about 24 hours after acute exposure to whole body ionizing radiation, wherein the low dose is less than about 400 ng/kg (15 μg/m²).

In another aspect, the present invention provides methods for increasing survival in a subject, increasing levels of erythropoietin (EPO) in subject, increasing levels of INF-γ in a subject, and/or preserving bone marrow function, and/or promoting hematopoietic recovery or restoration comprising the administration of an ultralow dose of IL-12 to the subject following an acute exposure to non-therapeutic whole body ionizing radiation. In one embodiment, an ultralow dose of IL-12 is used, such as a dose of less than about 3 μg/m². In another embodiment, the ultralow dose may be between about 300 ng/m² and about 2400 ng/m², or between about 600 ng/m² and about 1200 ng/m². In a particular embodiment, the low dose of IL-12 is about 900 ng/m².

In still yet another aspect, the present invention provides a method for increasing survival, increasing levels of erythropoietin (EPO) in subject, increasing levels of INF-γ in a subject, and/or preserving bone marrow function, and/or promoting hematopoietic recovery or restoration in a human comprising the administration of an ultralow dose of IL-12 to the subject following an acute exposure to non-therapeutic whole body ionizing radiation. In a specific embodiment, the method for treating a human comprises administering a low dose of IL-12 in a range between about 1 hour and about 24 hours after acute exposure to whole body ionizing radiation, wherein the low dose is less than about 80 ng/kg (3 μg/m²). The dose of IL-12 can also be, for example, less than about 100 ng/kg, less than about 250 ng/kg, or less than about 300 ng/kg,

In certain embodiments of the invention, a low or ultralow dose of IL-12 suitable for administration may be less than about or about 14 μg/m², less than about or about 13 μg/m², less than about or about 12 μg/m², less than about or about 11 μg/m², less than about or about 10 μg/m², less than about or about 9 μg/m², less than about or about 8 μg/m², less than about or about 7 μg/m², less than about or about 6 μg/m², less than about or about 5 μg/m², less than about or about 4 μg/m², less than about or about 3 μg/m², less than about or about 2 μg/m², less than about or about 1 μg/m², less than about or about 900 ng/m², less than about or about 800 ng/m², less than about or about 700 ng/m², less than about or about 600 ng/m², less than about or about 500 ng/m², less than about or about 400 ng/m², less than about or about 300 ng/m², less than about or about 200 ng/m², or less than about or about 100 ng/m².

In other embodiments, methods of multiple-dose IL-12 administration can comprise the administration of a first low or ultralow dose of IL-12, for example, a first dose of less than about or about 15 μg/m², less than about or about 14 μg/m², less than about or about 13 μg/m², less than about or about 12 μg/m², less than about or about 11 μg/m², less than about or about 10 μg/m², less than about or about 9 μg/m², less than about or about 8 μg/m², less than about or about 7 μg/m², less than about or about 6 μg/m², less than about or about 5 μg/m², less than about or about 4 μg/m², less than about or about 3 μg/m², less than about or about 2 μg/m², less than about or about 1 μg/m², less than about or about 900 ng/m², less than about or about 800 ng/m², less than about or about 700 ng/m², less than about or about 600 ng/m², less than about or about 500 ng/m², less than about or about 400 ng/m², less than about or about 300 ng/m², less than about or about 200 ng/m², or less than about or about 100 ng/m².

In one embodiment of the invention, the dosage of IL-12 is between about 100 ng/kg and about 250 ng/kg. In other embodiments of the invention, the dosage of IL-12 is about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, or about 400 ng/kg.

When administered in multiple doses, i.e. two, three, four, or more, the first IL-12 dose and subsequent IL-12 dose(s) can be equivalent doses, or they can be different dose amounts. For example, in certain embodiments, subsequent dose(s) can be administered at about 90% of the initial dose, or at about 80%, about 75%, about 70%, about 60%, about 50%, about 40%, about 30%, about 25%, about 20%, or about 10% or less of the original dose.

### C. IL-12 Compositions

In certain embodiments, the IL-12 is a mammalian IL-12, recombinant mammalian IL-12, murine IL-12 (rnIL-12), recombinant murine IL-12 (rmIL-12), human IL-12 (hIL-12), recombinant human IL-12 (rhIL-12), canine IL-12 or rIL-12, feline IL-12 or rIL-12, bovine IL-12 or rIL-12, equine IL-12 or rIL-12, or biologically active variants or fragments thereof. In one specific embodiment, the rhIL-12 is HemaMax™ (Neumedicines Inc.). In certain embodiments, the IL-12 can be modified in a fashion so as to reduce the immunogenicity of the protein after administration to a subject. Methods of reducing the immunogenicity of a protein are well known in the art and include, for example, modifying the protein with one or water soluble polymers, such as a PEG, a PEO, a carbohydrate, a polysialic acid, and the like.

It is well known that solutions of proteins that are formulated at low concentrations are susceptible to loss of a significant fraction of the protein prior to administration. One major cause of this problem is adsorption of the protein on the sides of tubes, vials, syringes, and the like. Accordingly, in certain aspects, when administered at low or ultralow doses, it will be beneficial to administer IL-12 along with a suitable carrier molecule or bulking agent. In one embodiment, the carrier agent may be a protein suitable for pharmaceutical administration, such as albumin. Generally, the carrier molecule or protein will be present in the formulation in excess of IL-12 to minimize the amount of IL-12 lost prior to administration. In certain embodiments, the carrier will be present at a concentration of at least about 2 times the concentration of IL-12, or at a concentration of at least about 3, at least about 4, at least about 5, at least about 6, at least about 7, at least about 8, at least about 9, at least about 10, at least about 25, at least about 50, at least about 100, or more times the concentration of IL-12 in the formulation.

IL-12 composition provided herein and used according to the methods of the invention can be formulated for administration via any known method, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Further, an efficacious dose of IL-12 may differ with different routes of administration.

In certain embodiment IL-12 is administered following radiation exposure by intramuscular (1M) or subcutaneous (SC) routes. Advantageously, these modes of administration can be self-administered or administered by a person with little or no medical training. As such, this aspect of the invention allows for rapid responses under disaster conditions where attention by those medical staff may be limited. Other modes of administration, however, such as intravenous and intraperitoneal, are also compatible with the present invention.

The IL-12 compositions can be administered in a variety of unit dosage forms depending upon the method of administration. For example, unit dosage forms suitable for oral administration include, but are not limited to, powder, tablets, pills, capsules and lozenges. It is recognized that constructs when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

In some embodiments, the formulations provided herein further comprise one or more pharmaceutically acceptable excipients, carriers, and/or diluents. In addition, the formulations provided herein may further comprise other medicinal agents, carriers, adjuvants, diluents, tissue permeation enhancers, solubilizers, and the like. Methods for preparing compositions and formulations for pharmaceutical administration are known to those skilled in the art (see, for example, REMINGTON'S PHARMACEUTICAL SCIENCES, 18^{TH} ED., Mack Publishing Co., Easton, Pa. (1990)). Formulations used according to the methods of the invention may include, for example, those taught in U.S. Pat. No. 5,744,132, which is hereby incorporated by reference in its entirety for all purposes.

### D. Tissue Expression of IL-12Rβ2

The IL-12Rβ2 protein is expressed in a variety of tissues in the human body outside of the hematopoietic system. These cell populations represent sources of tissue-specific progenitor cells that could also proliferate and reconstitute the cell population in the presence of IL-12. Exogenous IL-12 could also provide repair of damaged or diseased tissue. One example is damage caused by chemotherapy or radiation.

Immunohistochemistry for IL-12Rβ2 shows that the receptor subunit is strongly expressed in the brain, specifically neuronal cells of the cerebral cortex, hippocampus, lateral ventricle, and the cerebellum. See the Human Protein Atlas entry on the world wide web for “IL12RB2”. The Human Protein Atlas is described in Nat Biotechnol., 28(12):1248-50 (2010). Hepatocytes of the liver show weak staining for IL-12Rβ2, while glandular cells of the gall bladder exhibit moderate staining intensity. In the gastrointestinal tract, the stomach, duodenum, small intestine, appendix, and rectum show moderate staining for IL-12Rβ2. Other gastrointestinal regions show weak staining, such as the oral mucosa, salivary gland, esophagus, lymphoid tissue of the appendix, and the colon. Weak staining for IL-12Rβ2 has been shown in the nasopharynx, bronchus, and macrophages of the lung. Myocytes of the heart muscle also show weak staining. In the female reproductive system, the uterus shows moderate expression of IL-12Rβ2, while weak expression is exhibited in the breast, uterus, and fallopian tube. Decidual cells of the placenta show moderate expression of IL-12Rβ2, while trophoblastic cells of the placenta show weak expression. In the male reproductive system, moderate expression of IL-12Rβ2 is seen in leydig cells of the testis, while weak expression is seen in the epididymis, prostate, and seminal vesicle. Tubules in the kidney show moderate staining, while weak staining for IL-12Rβ2 is seen in the bladder. Skin also shows weak staining for IL-12Rβ2. Moderate IL-12Rβ2 staining is seen in the parathyroid and adrenal glands, while the thryoid exhibits weak staining for IL-12Rβ2.

### E. Exemplary Embodiments

In another aspect of the invention, methods are provided for increasing survival in a subject, increasing levels of erythropoietin (EPO) in subject, increasing levels of INF-γ in a subject, and/or preserving bone marrow function, and/or promoting hematopoietic recovery or restoration comprising the administration of more than one dose of IL-12 to a subject at protracted times following acute exposure to non-therapeutic whole body ionizing radiation. For example, in one embodiment, the method comprises the administration of a first therapeutically effective dose of IL-12 at a time up to about 24 hours post-irradiation and a second therapeutically effective dose of IL-12 at a second time up to about 72 hours after the first dose. In a particular embodiment, the first IL-12 dose can be administered between a range of about 1 hour and about 24 hours post-irradiation, or between a range of about 6 hours and about 24 hours post-irradiation, and the second IL-12 dose between a range of about 48 hours and about 1 week post-irradiation, or between a range of about 48 hours and about 94 hours post irradiation.

In one specific embodiment, the methods herein comprise administering a first dose of less than about 75 μg/m² IL-12 to a subject at a time between about 1 hour and about 24 hours after acute exposure to whole body radiation. In other embodiments, the method comprises administration of less than about or about 60 μg/m² IL-12, less than about or about 45 μg/m² IL-12, less than about or about 30 μg/m² IL-12, less than about or about 15 μg/m² IL-12, or less IL-12 at an effective time after acute exposure to radiation.

In one particular embodiment, a method is provided for increasing survival in a human, increasing levels of erythropoietin (EPO) in subject, increasing levels of INF-γ in a subject, and/or preserving bone marrow function, and/or promoting hematopoietic recovery or restoration, the method comprising the steps of administering a first dose of IL-12 to the human within about 24 hours following an acute exposure to non-therapeutic whole body ionizing radiation, wherein the first dose is less than about 100 μg/m² and subsequently administering a second dose of IL-12 to the human within about 96 hours following the acute exposure to non-therapeutic whole body ionizing radiation, wherein the second dose is less than about 100 μg/m². In a specific embodiment, the second dose is administered at least about 24 hours after the first dose is administered. In another specific embodiment, said second dose is less than the first dose.

In yet other embodiments, methods are provided for increasing survival in a subject, increasing levels of erythropoietin (EPO) in subject, increasing levels of INF-γ in a subject, and/or preserving bone marrow function, and/or promoting hematopoietic recovery or restoration following an acute exposure to non-therapeutic whole body ionizing radiation, comprising repeated administration of IL-12 for at least about a week, or at least about 2 weeks, or at least about 3, about 4, about 5, about 6, about 7, about 8, about 9, about 10, about 11, about 12, or more weeks. In certain embodiments, the doses may be administered about once every 12 hours, or about once every 24 hours, or about 1, about 2, about 3, about 4, about 5, about 6, or about 7 times a week. In other embodiments, IL-12 may be administered about every other week or about 1, about 2, about 3, about 4, about 5, or more times a month. In some embodiments, each IL-12 doses may be less than about 100 μg/m², or alternatively be an intermediate dose, a low dose, or an ultra low dose. In yet other embodiments, the doses may be decreased during the course of a repeated administration.

Without being held to any particular theory, it is believed that when the hematopoietic system is compromised, as with acute whole body radiation, the IL-12 mediated pathway leading to the production of INF-γ may be sensitized. Consistent with this, Hixon et al. observed that when IL-12 is administered to mice following whole body irradiation and bone marrow transplant, INF-γ levels are greatly increased and acute lethal toxicity of the gut results from the elevated levels of INF-γ. The observed acute lethal toxicity of the gut was dependant on INF-γ, as INF-γ knockout mice were resistant to IL-12 mediated toxicity. The experiments performed by Hixon et at. support a model in which IL-12 administration results in an increase of INF-γ levels, and therefore an increase in Crg-2 and Mig cytokine levels, resulting in anti-angiogenesis effects. Alternatively, but not mutually exclusive with the above, by performing bone marrow transplants prior to IL-12 administration, Hixon et al. provided INF-γ producing lymphocytes to the mice, further increasing the potential for INF-γ production.

Thus, a possible mechanism for the decreased hematopoietic side effects associated with certain embodiments of the invention is that when a relatively low dose IL-12 is given to a mammal whose hematopoietic system is compromised, the dose is insufficient to upregulate INF-γ production. Since INF-γ inhibits hematopoiesis and also appears to be the major cytokine responsible for toxicity, the absence of INF-γ upregulation upon administration of low and ultralow doses of IL-12, as used in the methods of the present invention, may be one of the factors underlying the discovery by the inventors that administration of low and ultralow doses of IL-12 provides a hematopoietic protective and recovery effect without apparent toxicity.

Also of significance is the demonstration that exogenous administration of IL-12 an expand long-term repopulating (LTR) hematopoietic stem cells (HSC) in vivo. Thus, without being bound by theory, HSC expansion by exogenous IL-12 can be the mechanism responsible for survival from hematopoietic injury resulting from lethal radiation exposure at later time points, e.g., 24 hours post-irradiation. Another potential mechanism relates to the ability of IL-12 to induce DNA repair and reduce apoptosis in hematopoietic stem cells (HSC) following radiation exposure.

While most bone marrow progenitor and stem cells are susceptible to cell death after high dose radiation, subpopulations of HSC or accessory cells are selectively more radioresistant, presumably because these cells exist in a largely noncycling (Go) state. In humans, these radioresistant cells can play an important role in recovery of hematopoiesis after exposure to doses as high as 6 Gy, albeit with a reduced capacity for self-renewal in the absence of exogenous IL-12.

Another determinant for hematopoietic reconstitution is non-homogeneity of the radiation dose, which can spare some marrow sites that then become the foci of hematopoietic activity. In either case, i.e., either the residual presence of radioresistant HSC or inhomogeneity of the radiation dose, the present findings indicate that a subpopulation of HSC marked by the presence of the IL-12 receptor (IL-12R+) survives and persists after high dose radiation, and moreover, that this IL-12R+ HSC subpopulation is activated, expanded, and/or induced to repair itself upon exogenous administration of IL-12.

Following radiation exposure, it has been discovered that IL-12 is effective in mitigating the hematopoietic syndrome associated with acute radiation syndrome. Specifically, embodiments of the present invention provide methods for increasing survival, and/or preserving bone marrow function, and/or promoting hematopoietic recovery by administering one or more effective dose(s) of IL-12 to a subject following acute exposure to ionizing radiation.

For humans, as shown in Table 2, the early signs of hematopoietic syndrome start to occur in the range of radiation doses of 2 Gy or greater. Similarly, at radiation doses between about 5.5-7.5 Gy, pancytopenia and moderate 01 damage occurs in humans. Advantageously, when administered according to the methods of the present invention, IL-12 is effective in alleviating the pancytopenia at these radiation dose levels, preserving bone marrow function and will not induce further GI damage. However, the radiation dose rate can also affect the relative level of radiation injury. Thus, two radiation doses given at two different dose rates can show differences in the severity of the relative radiation injury.

TABLE 2
Phases of Radiation Injury*

| Dose Range | | | |
|---|---|---|---|
| Gy | Prodrome | Manifestation of Illness | Prognosis (without Therapy) |
| 0.5-1.0 | Mild | Slight decrease in blood cell counts | Almost certain survival |
| 1.0-2.0 | Mild to moderate | Early signs of bone marrow damage | Highly probable survival (>90% |
| | | | of victims) |
| 2.0-3.5 | Moderate | Moderate to severe bone marrow | Probable survival |
| | | damage | |
| 3.5-5.5 | Severe | Severe bone marrow damage, slight | Death within 3.5-6 wk (50% of |
| | | GI damage | victims) |
| 5.5-7.5 | Severe | Pancytopenia and moderate GI | Death probable within 2-3 wk |
| | | damage | |
| 7.5-10.0 | Severe | Marked GI and bone marrow | Death probable within 1-2.5 wk |
| | | damage, hypotension | |
| 10.0-20.0 | Severe | Severe GI damage, pneuomonitis, | Death certain within 5-12 d |
| | | altered mental status, cognitive | |
| | | dysfunction | |
| 20.0-30.0 | Severe | Cerebrovascular collapse, fever, | Death certain within 2-5 d |
| | | shock | |

*Modified from Walker RI, Cerveny RJ, eds. (21),
GI = gastrointestinal

For other mammals embraced by the methods and compositions of the present invention, for example mice, rats, guinea pigs, hamsters, cats, dogs, cattle, horses, sheep, pigs, rabbits, deer, monkeys, and the like, the radiation dose that can induce hematopoietic syndrome varies with the species and strain. For example, for rhesus monkeys, the LD₅₀ is about 7 Gy. For certain strains of mice, the LD₅₀ is also about 7 Gy, for example Balb-c mice. For other strains of mice, such as C57BL6, the LD₅₀ is about 7.5 Gy. The LD₅₀ can also exhibit differences based on gender or general health status of the animal.

Although it would be difficult to determine the exact extent of radiation injury in a mammal exposed to acute ionizing radiation following a radiation-related disaster, IL-12, when used in accordance with embodiments of the present invention, will increase survival, and/or preserve bone marrow function, and/or promote hematopoietic recovery of peripheral blood cell counts.

Accordingly, in some embodiments of the present invention, IL-12 is administered to a subject that has been exposed to an acute dose of ionizing radiation of at least about 1.0 Gy, or an amount equivalent to an LD₁₀ in humans. In another embodiment, IL-12 is administered to a subject that has been exposed to about 3.5 Gy of ionizing radiation, or a dose equivalent to about LD₅₀ in humans. In yet other embodiments, IL-12 is useful for increasing survival, and/or preserving bone marrow function, and/or promoting hematopoietic recovery of peripheral blood cell counts in a subject exposed to at least about 1.0 Gy, at least about 2.0 Gy, at least about 3.0 Gy, about 4.0 Gy, about 5.0 Gy, about 6.0 Gy, about 7.0 Gy, about 8.0 Gy, about 9.0 Gy, about 10.0 Gy, about 11.0 Gy, about 12.0 Gy, about 13.0 Gy, about 14.0 Gy, about 15.0 Gy, about 20.0 Gy, about 25.0 Gy, about 30.0 Gy, or higher doses of acute ionizing radiation. Similarly, the dose of ionizing radiation can be expressed in terms of the percent lethal dose, for example, a dose equivalent of about LD₁, about LD₅, about LD₁₀, about LD₂₀, about LD₃₀, about LD₄₀, about LD₅₀, about LD₆₀, about LD₇₀, about LD₈₀, about LD₉₀, about LD₉₅, about LD₉₉, or about LD₁₀₀.

In another embodiment of the invention, acute exposure to whole body ionizing radiation is the result of a nuclear event. The acute exposure can be exposure to at least about 1.0 Gy, at least about 1.5 Gy, at least about 2.0 Gy, at least about 2.5 Gy, at least about 3.0 Gy, at least about 3.5 Gy, at least about 4.0 Gy, at least about 4.5 Gy, at least about 5.0 Gy, at least about 5.5 Gy, at least about 6.0 Gy, at least about 6.5 Gy, at least about 7.0 Gy, at least about 7.5 Gy, at least about 8.0 Gy, at least about 8.5 Gy, or at least about 9.0 Gy.

As shown in the Examples below, administration of IL-12 following an acute exposure to non-therapeutic whole body ionizing radiation can increase survival by up to about 90%, as compared to survival of less than 15% when exposed to the same level of radiation in the absence of IL-12 administration. In one embodiment, survival is increased with IL-12 administration post-radiation exposure by at least about 10%, at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 100%, as compared to survival in the absence of IL-12 administration.

In another embodiment of the invention, the subject's survival rate following an acute exposure to non-therapeutic whole body ionizing radiation is at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%.

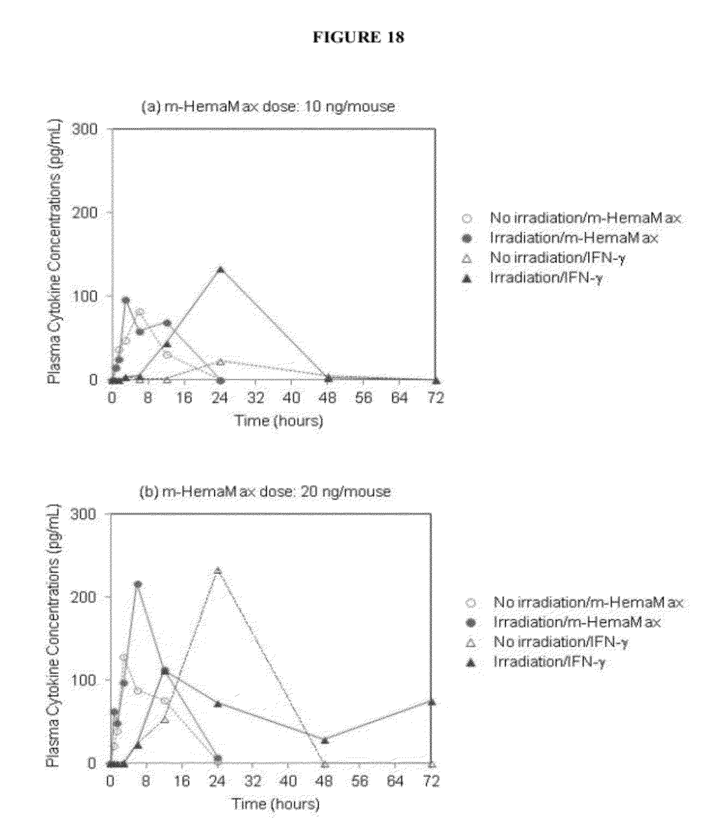

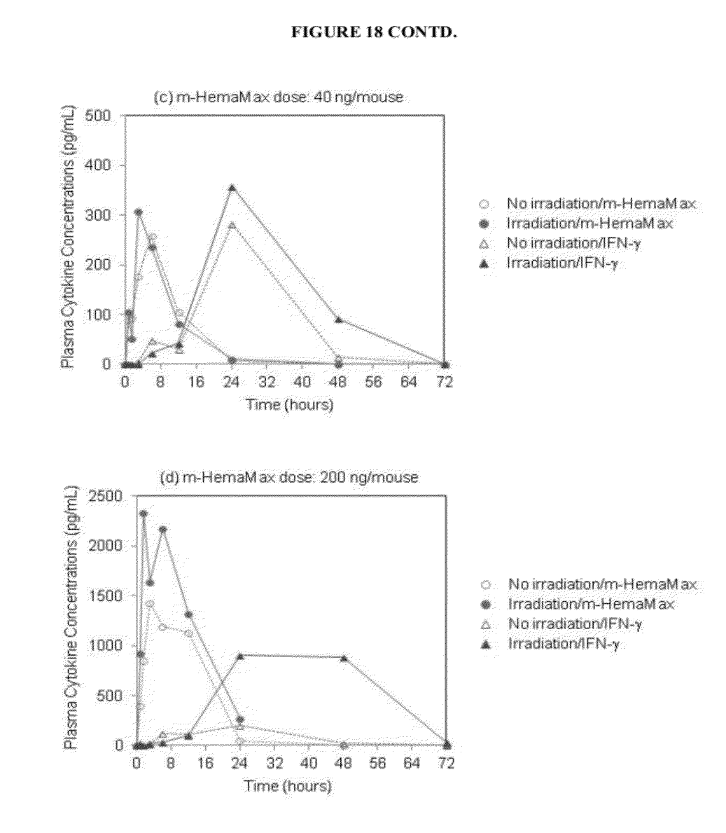

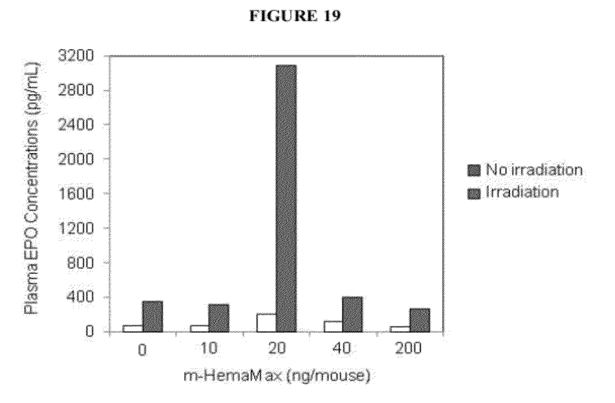

Also as detailed in the examples below, at radiation doses where immune, bone marrow, and GI damage overlap, IL-12 provides mitigation of injury in all three radiosensitive tissues, thereby leading to an increase in survival that is independent of radiation dose within a certain window of exposure. Moreover, the data in the examples show that increased plasma EPO levels may play a role in the suppression of plasma IFN-γ levels in irradiated mice that received to IL-12 at the dose of 20 ng/mouse (see FIG. 18B), leading to a decrease in the inflammatory response to radiation. Furthermore, the data in the examples reveal that there is a window of opportunity for mitigation of radiation injury by IL-12 in a very low dose range that is also effective in alleviating bone marrow damage. The data in the examples also show that IL-12-induced increase in blood cell counts around nadir play a key role in promoting survival following radiation exposure.

### F. Supportive Care

In another aspect of the invention, methods are provided for increasing the probability of survival in a subject, increasing levels of erythropoietin (EPO) in subject, increasing levels of INF-γ in a subject, and/or preserving bone marrow function, and/or promoting hematopoietic recovery or restoration comprising the administration of one or more dose of IL-12 to a subject at protracted times following acute exposure to non-therapeutic whole body ionizing radiation, wherein supportive care is given to the subject simultaneously or following administration of IL-12.

Supportive care modalities useful in conjunction with IL-12 for treatment of a subject who has been exposed to an acute dose of whole body ionizing radiation include, without limitation, administration of fluids, one or more antibiotics, blood or blood component transfusions, administration of one or more growth factors or hematopoietic growth factors, combination therapies and the like.

In one embodiment, supportive care comprises the administration of one or more antibiotics. Antibiotic support can be any antibiotic that is useful in preventing infections during periods of low blood cell counts including, without limitation, bactrim, ciprofloxacin, moxifloxacin, and the like. Those of skill in the art will know of other antibiotics useful for supportive care.

In another embodiment, supportive care comprises administration of one or more growth factors, including hematopoietic growth factors. Many suitable hematopoietic growth factors are known in the art including, without limitation, colony stimulating factors (CSF, G-CSF, GM-CSF, M-CSF, IL-3), erythropoietin, IL-1, IL-4, IL-5, IL-6, IL-7, IL-11, and the like. Several FDA-approved hematopoietic growth factors are currently available, and thus may be used in the methods provided herein, such as G-CSF (Neupogen or Neulasta), IL-11, and erythropoietin (Epogen, Procrit or Aranesp). In some embodiments, supportive care comprises the administration of keratinocyte growth factor (KGF or FGF7).

In one particular embodiment, erythropoietin administration can increase survival up to about 50% over and above that of IL-12 alone when super-lethal doses are used with no other supportive care measures, such as antibiotic support or fluid administration. Superlethal doses are defined herein as radiation doses at or above 5.5 Gy. Erythropoietin is available as a FDA-approved recombinant protein drug for human use, such as Epogen, Procrit or Aranesp. Generally, dosing with these erythropoietin drugs will be simultaneous with or following the administration of IL-12. Erythropoietin drugs can be repeated as needed, but generally not administered more than every other day, or every third day. Preferably erythropoietin is administered about 48 hours after the last dose of IL-12.

An effective dose of erythropoietin for a human can be about 20 mg/kg, however, lower doses and higher doses are also effective in increasing survival when use as an adjuvant to IL-12 administration. Accordingly, in certain embodiments, erythropoietin is administered at about 1 mg/kg, or at about 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, or more for treatment in a human, or at a dose equivalent amount for treatment in an animal other than a human.

In another embodiment, supportive care comprises the administration of a blood transfusion. As used herein, a blood transfusion may encompass a whole blood transfusion, or alternatively, transfusion of a blood fraction or blood component, for example, a red blood cell transfusion, a platelet transfusion, a white blood cell transfusion. In a related embodiment, supportive care may comprise the administration of a bone marrow or bone marrow stem cell transplant.

### G. Kits

In another aspect of the invention, kits are provided for increasing survival in a subject, increasing levels of erythropoietin (EPO) in subject, increasing levels of INF-γ in a subject, and/or preserving bone marrow function, and/or promoting hematopoietic recovery or restoration following non-therapeutic acute exposure to whole body ionizing radiation. In one embodiment, a kit of the invention comprises one or more formulations of a therapeutically effective amount of IL-12. In certain embodiments, the therapeutically effective amount of IL-12 comprises a low dose of IL-12 described herein, for example, a dose that is less than about 15 μg/m². In other embodiments, the therapeutically effective amount of IL-12 may comprise an ultralow dose of IL-12 described herein, for example, a dose that is less than about 3 μg/m². In yet other embodiments, a kit as provided herein may comprise multiple doses or formulations suitable for multi-dose administration of IL-12 following radiation exposure.

In one specific embodiment, a kit is provided for increasing survival in a human, increasing levels of erythropoietin (EPO) in subject, increasing levels of INF-γ in a subject, and/or preserving bone marrow function, and/or promoting hematopoietic recovery or restoration following non-therapeutic acute exposure to whole body ionizing radiation, comprising a low dose or ultralow dose of human IL-12. In one embodiment, the human IL-12 is recombinant IL-12 or a recombinant IL-12 variant. In the kits of the invention, IL-12 can be formulated so as to be delivered to a patient using a wide variety of routes or modes of administration. In a particular embodiment, IL-12 is formulated for injection in solution or as a lyophilized powder that can be easily reconstituted using sterile water or a physiologically acceptable buffer.

In some embodiments, the kits of the invention may also comprise a formulation of a compound useful for supportive care of IL-12 treatment following radiation exposure. Suitable compounds include, without limitation, antibiotics, for example, bactrim, ciprofloxacin, or moxifloxacin, and hematopoietic growth factors, such as CSF, G-CSF, GM-CSF, M-CSF, IL-3, erythropoietin, erythropoietin-like molecules, IL-I, IL-4, IL-5, IL-6, IL-7, IL-11, and the like.

In certain embodiments, kits are provided for treating one or more animal following acute exposure to whole body ionizing radiation. For example, kits are provided for treating one or more of a human, dog, cat, guinea pig, hamster, cattle, horse, sheep, pig, rabbit, and the like. In a particular embodiment, these kits contain one or more dose of IL-12 specific for the animal to be treated. For example, a kit for treating cattle may comprise bovine IL-12, or a kit for treating horses may comprise equine IL-12. In other embodiments, the kits comprise, for example, murine or human IL-12 at a dose suitable for administration to the particular animal to be treated.

In certain aspects, the kits provided herein are optionally housed in a radiation proof container (e.g., lead) or alternatively, the container for the kit is radiation resistant or proof (e.g., lead).

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### III. Summary of IL-12 Effects

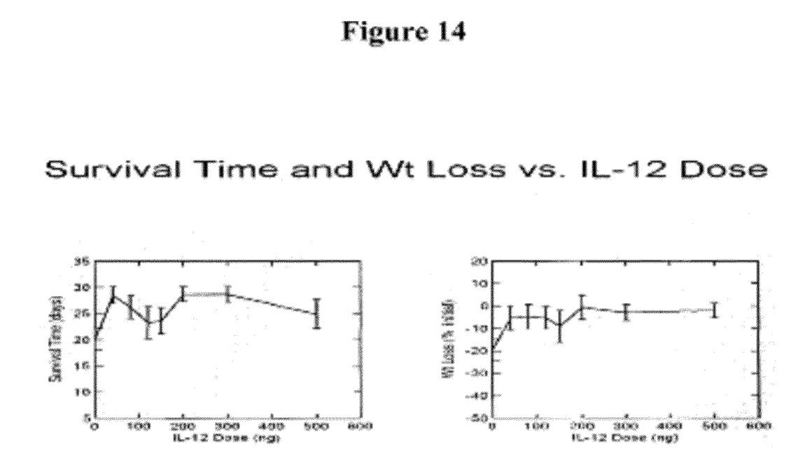

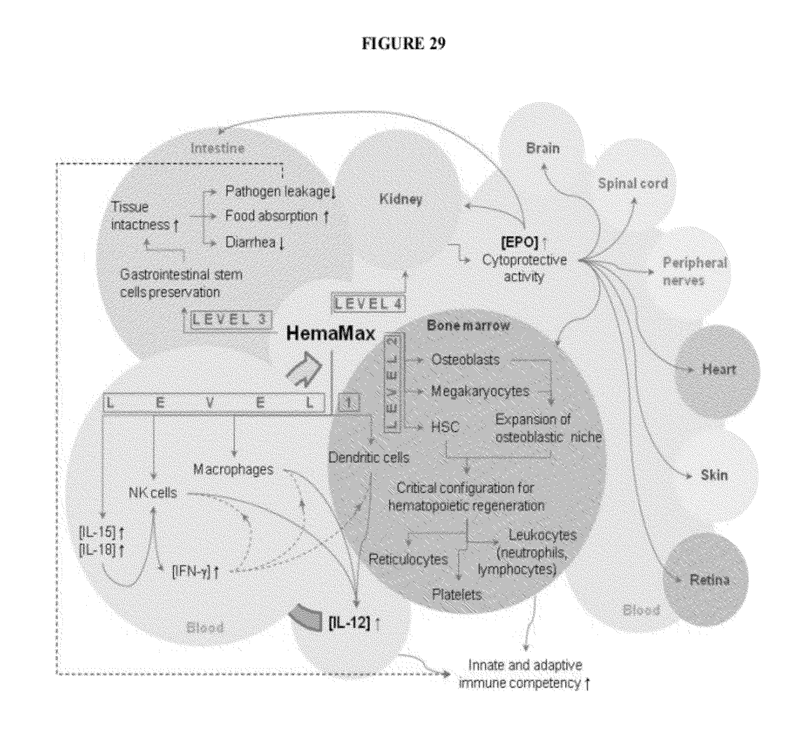

The studies disclosed herein suggest a multilevel response orchestrated by exogenous delivery of recombinant IL-12 (HemaMax™) (see FIG. 14). HemaMax™ triggers responses in at least 4 general types of cells, or “levels”, by directly activating IL-12 receptors (a) on immune cells in peripheral blood and bone marrow (Level 1), (b) on hematopoietic stem cells and other key cells of the bone marrow niche, such as osteoblasts (Level 2), (c) on GI stem cells (Level 3), and likely (d) on kidney cells (Level 4), whereby EPO, a cytoprotective factor, is released following radiation exposure (see FIG. 29).

At the Level 1 response, HemaMax™ promotes proliferation and activation of extant, radiosensitive immune cells, namely NK cells, macrophages, and dendritic cells. HemaMax™-induced plasma elevations of IL-15 and IL-18 also facilitate maturation of NK cells, leading to the release of IFN-γ, which in turn, positively affects the production of endogenous IL-12 from macrophages and dendritic cells, and perhaps NK cells. These events enhance the innate immune competency early on following HemaMax™ administration. At the Level 2 response, HemaMax™ promotes proliferation and differentiation of the surviving hematopoietic stem cells, osteoblasts, and megakaryocytes into a specific cellular configuration that ensues optimal hematopoiesis. HemaMax™-induced secretion of EPO from CD34+, IL-12Rβ2—positive bone marrow cells may also suppress local over-production of IFN-γ in the bone marrow and, thus, provide a milieu that promotes expansion of hematopoietic cells. Hematopoietic regeneration in the bone marrow enhances both innate and adaptive immune competency. At the Level 3 response, HemaMax™ preserves GI stem cells, leading to a reduction in pathogen leakage, an increase in food consumption, and a decrease in diarrhea. At the Level 4 response, HemaMax™ likely directly increases renal release of EPO, a cytoprotective factor, which enhances cellular viability in a diverse set of organs/tissues. Continued production of endogenous IL-12 primarily from dendritic cells activated by pathogens and/or EPO serves as a positive feedback loop and plays a key role in sustaining the initial response to exogenous HemaMax™, perhaps for weeks after radiation.

### IV. Examples

### Example 1

### The IL-12-Facilitated Survival as a Function of Administration Schedule Following Super-Lethal Radiation (9 Gy)

This example illustrates the effects of administration time point of IL-12 in a comparative experiment, where various time points were directly compared to assess relative survival and hematopoietic recovery. IL-12 was administered at 3 hours after, 24 hours before, or 24 hours before and 2 hours after (at two different doses of IL-12) using a 9 Gy radiation dose (unfractionated dose at a dose rate of 0.9 Gy/min (γ-ray from cesium 137 irradiator). In this example, female C57BL/6 mice were used (13 weeks of age). All injections were intravenous, and mice were started on antibiotics (Bactrim) following radiation.

As shown in Table 3, survival data demonstrate that there was not significant differences among the various time points evaluated. In fact, administration of IL-12 at 3 hours after radiation or 24 hours before radiation showed only a slight difference in survival, albeit the two groups were administered a different dose of IL-12. However, it was determined that 100 ng (15 μg/m²) is the optimal dose of IL-12 when given 24 hours before lethal radiation. (See Table 3). Moreover, at the same overall dose of IL-12 (200 ng/mouse; 30 μg/m2) given either in a split dose of given at 24 hour before and 2 hours after radiation or as a single dose given at 3 hours after radiation, there was no difference in relative survival. These data suggest that a residual subpopulation of hematopoietic stem cells persist after radiation that can be acted upon by IL-12.

TABLE 3
Survival as a Function of Time of Administration of IL-12 at 9 Gy
Acute Radiation Dose.

| Time of Administration | Dose of IL-12 | % Survival |
|---|---|---|
| PBS control | 0 | 0 |
| 3 hours after radiation | 200 ng (30 μg/m²) | 60 |
| 24 hour before radiation | 100 ng (15 μg/m²) | 50 |
| 24 hr before and 2 hr after | 100 ng, 50 ng | 70 |
| | (15 μg/m², 7.5 μg/m²) | |
| 24 hr before and 2 hr after | 100 ng, 100 ng | 60 |
| | (15 μg/m², 15 μg/m²) | |

### Example 2

### The IL-12-Facilitated Hematopoietic Recovery as a Function of Administration Schedule Following Super-Lethal Radiation (9 Gy)

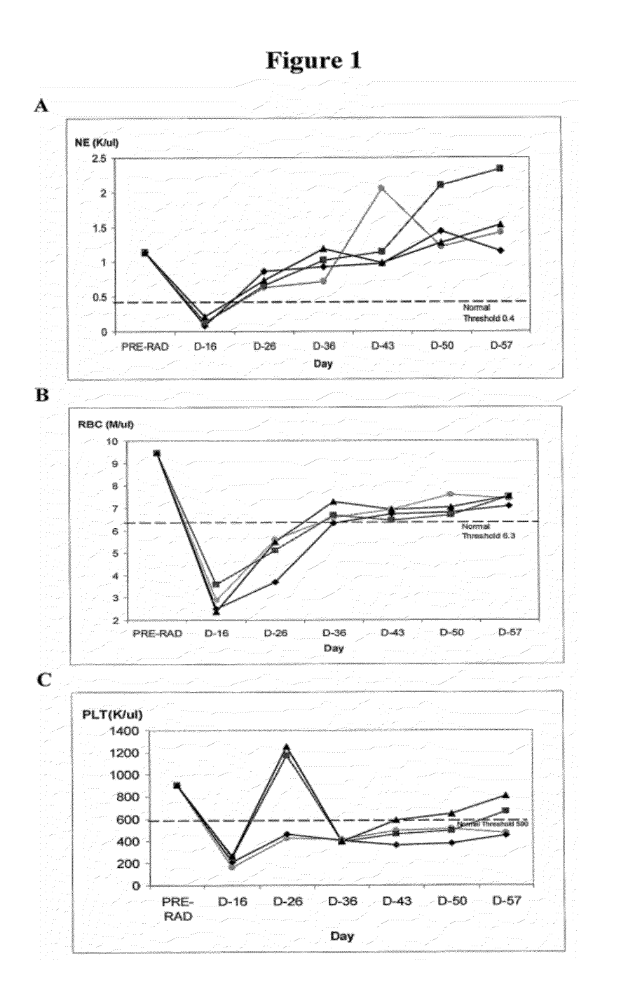

The peripheral blood recovery for mice that survived the super-lethal radiation dose is shown in FIG. 1. Although there are some notable difference in the neutrophil and red blood cell recovery, overall the recovery of these two blood cell groups was quite similar, and did not vary significantly as a function of the time point of IL-12 administration. Platelet recovery, however, was significantly different for the 3 hour post- and 24 hour pre-radiation administration of IL-12, as compared to the split dose (pre-post) administration. For this example, mice were administered Bactrim in their drinking water, which is known to induce thrombocytopenia. Mice were taken off antibiotics at about 48 days, and subsequently, all groups showed an increase in platelet counts. These results show that IL-12 can facilitate recovery of all blood cell groups, including potent recovery of platelet counts, at super-lethal radiation dose (9 Gy TBI). Also notable is the length of the survival time. Mice were monitored for 57 days and were terminated on day 60. AU mice appeared in good health and had regained any losses in body weight.

### Example 3

### IL-12-Facilitated Survival: Administration of IL-12 at 6 Hours after Lethal Radiation (LD_{100/10}) to Female Mice without Antibiotic Support

In this example, the effect of IL-12 when administered at 6 hours after an unfractionated (acute), lethal dose of radiation (8 Gy) without the addition of antibiotic support was evaluated.

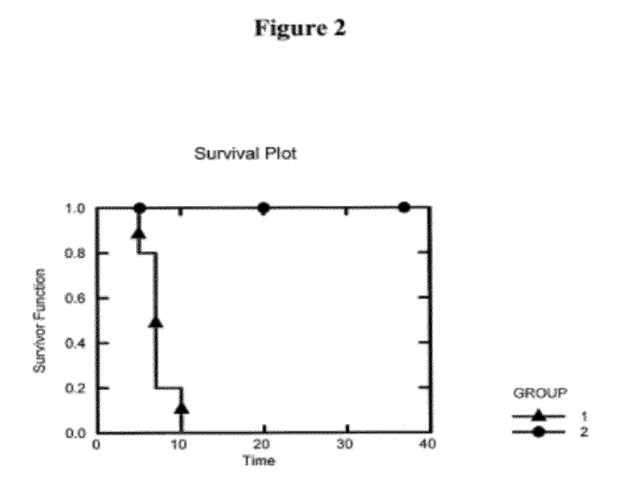

A Kaplan-Meier plot of the data is shown in FIG. 2. Remarkably, at the LD_{100/10}, 100% of the IL-12-treated mice at 6 hour post radiation survived. Moreover, the health status of the IL-12-treated mice was remarkably stable during the period in which control mice experienced the effects of hematopoietic syndrome.

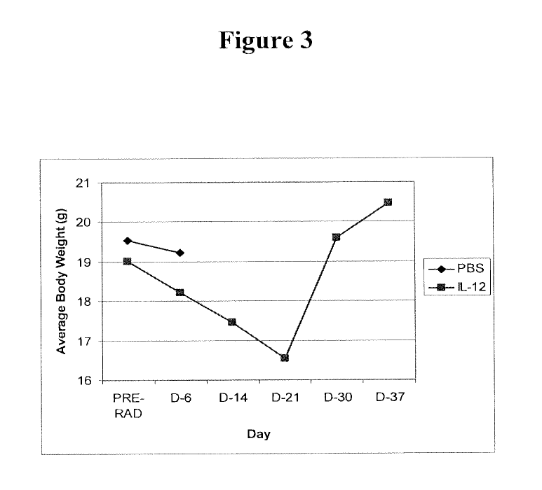

As shown in FIG. 3, although the IL-12-treated mice did lose about 14%, on average of their body weight by day 21 post radiation, they had regained all of their lost body weight, and were slightly higher in weight than they were at the start of the experiment, by about day 30 (19.1 g on day 0 vs. 19.6 g on day 30).

As would be expected, statistical analyses revealed that the survival effect was highly significant via the Mantel Chi-Square statistical method (p=0.001), despite the small number of mice (n=5 for Group 1 and n=6 for Group 2). This example demonstrates the remarkable survival effects of IL-12 at 6 hours post radiation.

### Example 4

### IL-12-Facilitated Survival: Administration of IL-12 at 6 Hours after Lethal Radiation (LD_{100/21}) Using Male Mice and No Antibiotic Support

This example evaluated the effect of IL-12 when administered at 6 hours after an unfractionated (acute), lethal dose of radiation (8 Gy) in male mice without the addition of antibiotic support. This example shows a lethal radiation survival study using male mice.

Two groups of mice were first exposed to a 7 Gy dose of radiation and either treated with IL-12 or vehicle (PBS) at about 9 weeks of age. For the first round of radiation, Group 1 received PBS and Group 2 received IL-12, all mice survived following radiation. After 5 weeks, when it was clear that all mice were healthy enough to undergo a second round of radiation, the same mice were then subjected to a radiation dose of 8 Gy. At this point, the mice were about 14 weeks of age and weighed 27 grams on average. The IL-12 was adjusted according to the weight of the mice. For the second round of radiation, Group 1 again received only PBS and Group 3 also received PBS, and Group 2 received IL-12 again.

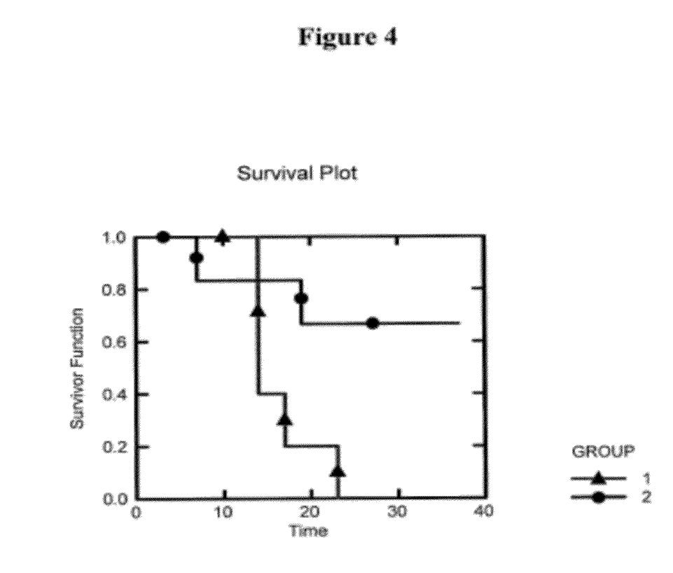

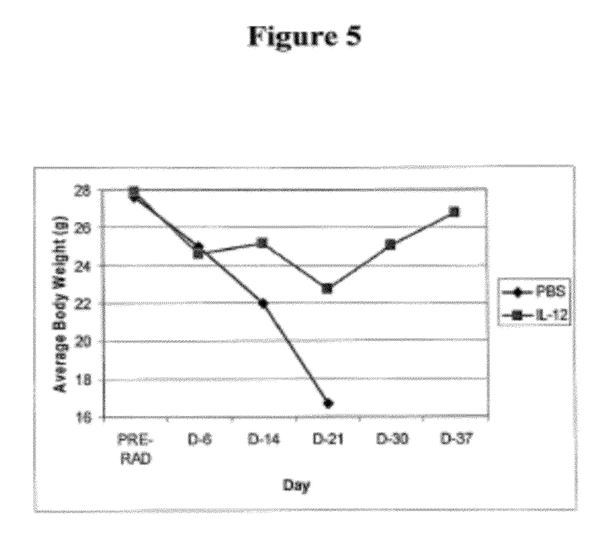

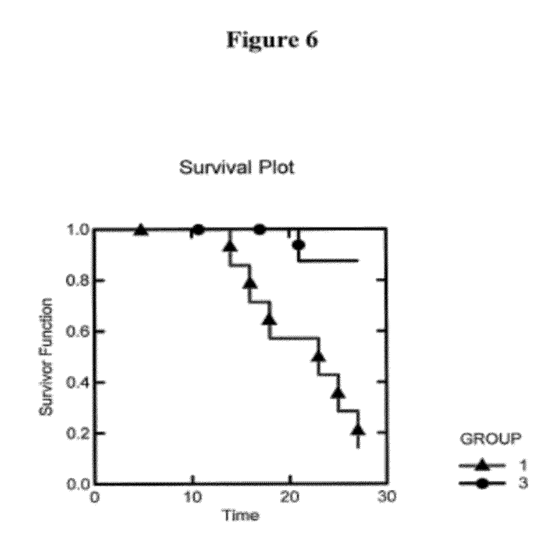

A Kaplan-Meier plot of the data is shown in FIG. 4 (Groups 1 (PBS) vs. 2 (IL-12)). Remarkably, it was found that after receiving an accumulated dose of 15 Gy, nearly 70% of the IL-12-treated mice survived (Group 2) when IL-12 was administered at 6 hours post radiation. Moreover, the health status of the IL-12-treated mice in Group 2 was observed to be good during the entire observation period for the surviving mice. During the second round of radiation, surviving mice lost about 24% of their body weight by day 21 post radiation, and on day 30 these mice had regained 10% of their body weight. These results are shown in FIG. 6. Remarkably, statistical analyses revealed that the survival effect was significant via the Chi-Square statistical Mantel method (p<0.05), despite the small number of mice (n=5 for Group 1 and n=6 for Group 2).

The data presented in FIGS. 3 and 4 were collected at the same time, i.e., the mice were all irradiated at the same time and received the same radiation dose. However, there is clearly a notable difference in the survival curve for control female mice as compared to control male mice. This may be due to apparent differences in intrinsic radiation rescue response for males and females, such as sex differences in endogenous IL-12 production following radiation or other related factors.

### Example 5

### IL-12-Facilitated Survival: Administration of IL-12 at 24 Hours after Lethal Radiation (LD_{90/30}) Using Female Mice and No Antibiotic Support

In this example, IL-12 administration was evaluated when administered at 24 hours after an unfractionated (acute), lethal dose of radiation (8 Gy) without the addition of antibiotic support.

A Kaplan-Meier plot of the data is shown in FIG. 6. Remarkably, when IL-12 was administered at 24 hours after an 8 Gy radiation dose, 90% of the IL-12 treated mice survived, whereas only 14% of the control mice were alive at 27 days post radiation. Moreover, the health status of the IL-12-treated mice was remarkably stable during the period in which control mice experienced the effects of hematopoietic syndrome.

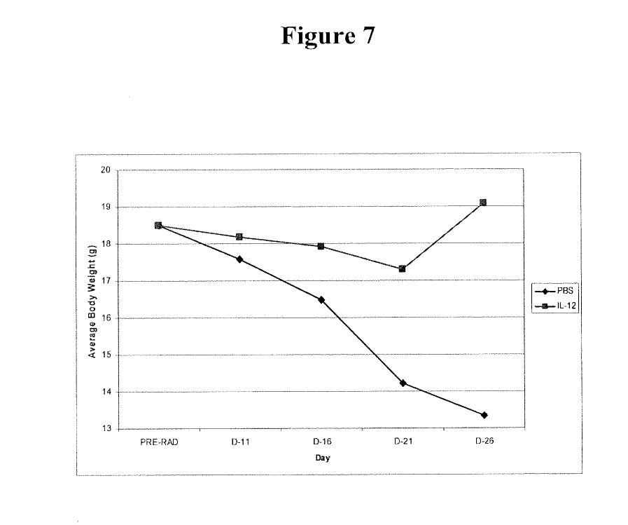

As shown in FIG. 7, although the IL-12-treated mice did lose about 10%, on average, of their body weight by day 21 post radiation, they had regained all of their lost body weight, and were slightly higher in weight than they were at the start of the experiment, by about day 27 (18.5 g on day 0 vs. 19.2 g on day 27).

Statistical analyses revealed that the survival effect was significant via the Mantel Chi-Square statistical method (p=0.001), despite the small number of mice (n=7 for Group 1 and n=8 for Group 2). This example demonstrates the remarkable survival effects of IL-12 at 24 hours post radiation.

### Example 6

### Low Dose Administration of IL-12 at 24 Hours and at 24 Hours and 72 Hours after a Lethal Dose of Acute Whole Body Irradiation (LD₈₀)

TABLE 4
Experimental design for single dose and double dose IL-12
administration after acute whole body irradiation.

| | | |
|---|---|---|
| | muIL-12 dose | muIL-12 dose |
| Group | (ng per mouse) | (μg/m²) |

4A: Double Dose Experiment*

| | | |
|---|---|---|
| 1 | 0 (vehicle) | 0 |
| 2 | 40 | 6 |
| 3 | 80 | 12 |
| 4 | 120 | 18 |
| 5 | 150 | 22.5 |
| 6 | 200 | 30 |
| 7 | 300 | 45 |
| 8 | 500 | 75 |

4B: Single Dose Experiment*

| | | |
|---|---|---|
| 1 | 0 (vehicle) | 0 |
| 2 | 40 | 6 |
| 3 | 80 | 12 |
| 4 | 120 | 18 |
| 5 | 150 | 22.5 |
| 6 | 200 | 30 |
| 7 | 300 | 45 |
| 8 | 500 | 75 |

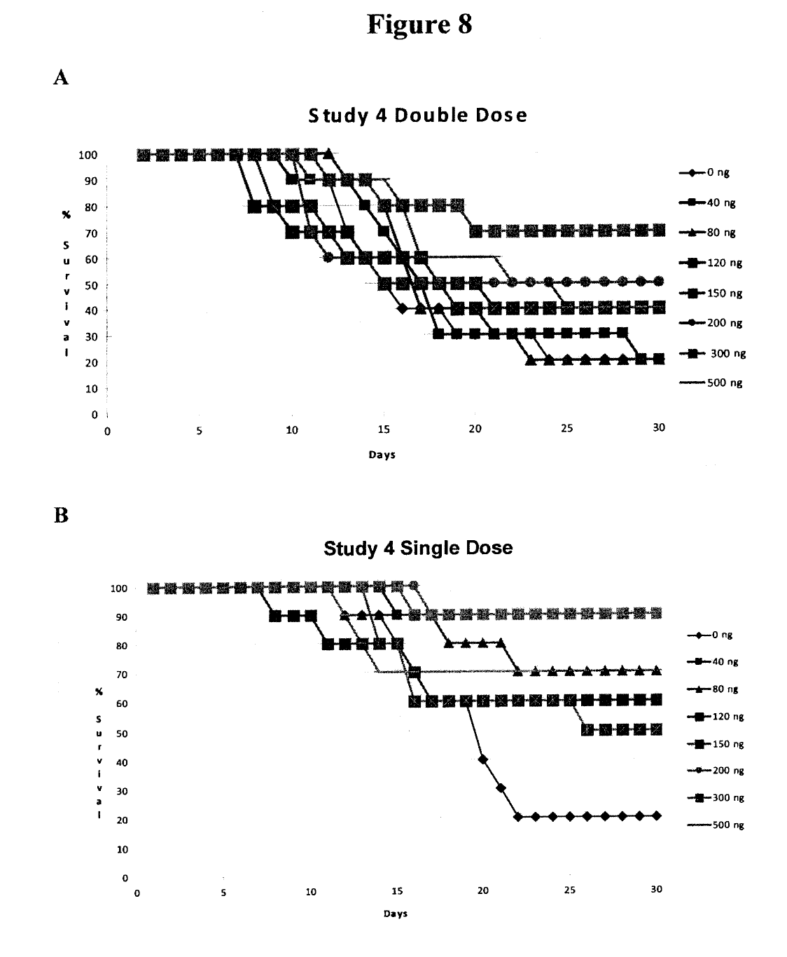

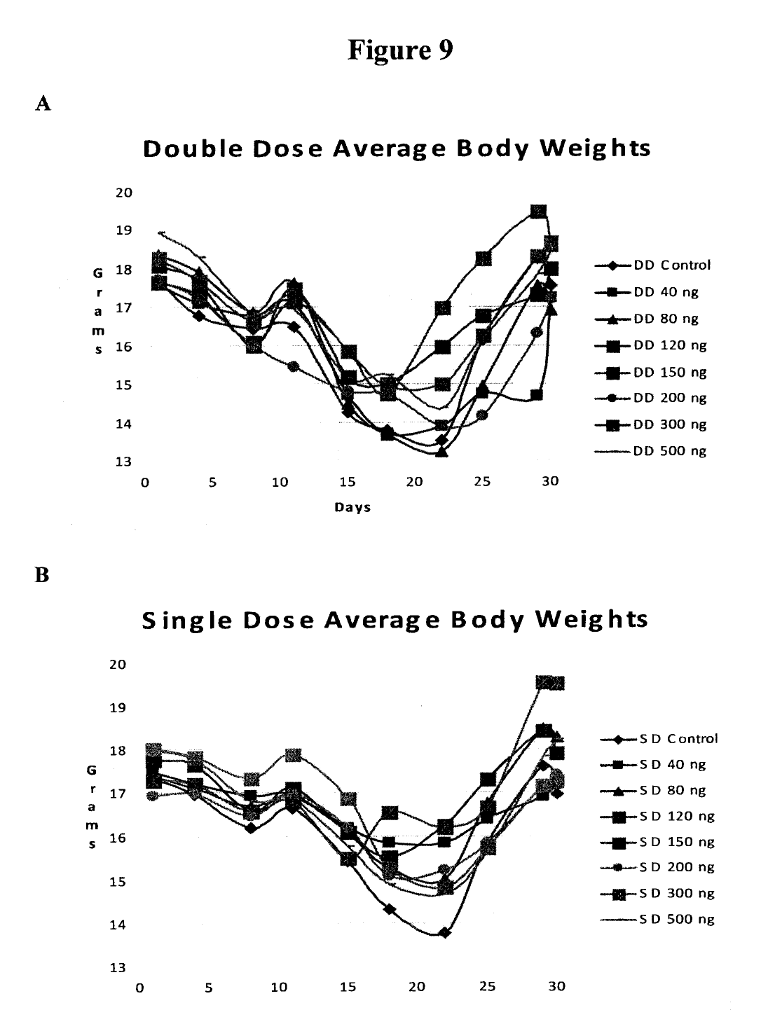

Mice treated with a double dose of IL-12 showed a linear dose response related to administration of muIL-12 at lethal radiation doses 24 and 72 hours post radiation (FIG. 8A), whereas studies of single dose treatments demonstrate a biphasic dose response related to administration of muIL-12 at lethal radiation doses 24 hours post radiation (FIG. 8B). For the double dose experiment, higher doses of muIL-12 gave better efficacy, as measured by survival following a LD₈₀ radiation dose with the most efficacious dose being 300 ng (45 μg/m²) given twice. Interestingly, for the single dose experiments, the dose response curve was biphasic with 40 ng (6 μg/m²) and 200-300 ng (30 μg/m²-45 μg/m²)) showing high efficacy at the same LD₈₀₁₃₀ radiation dose. Average body weights for the surviving mice are shown in FIG. 9.

The efficacy at 300 ng (45 μg/m²) in the double dose experiment was 70% at the LD_{80/30} radiation dose (FIG. 8A). Notably, in the double dose experiment, there were early deaths in many of the IL-12 treated groups, as compared to the control. Without being bound by theory, these early deaths may be due to protein aggregation resulting in immunogenicity. Consistent with this notion, there were very few early deaths in the single dose experiment. As can be seen via the statistical evaluation below, in the double dose experiment it appears that there may be two effects: 1) a highly therapeutic effect and 2) an effect that diminishes the efficacy of the therapeutic effect.

The results of the single dose experiments show that a single low dose of IL-12 (40 ng; 6 μg/m²) administered at 24 hours after acute whole body irradiation resulted in 90% survival at the LD_{80/30} (FIG. 8B). This single dose gave a 70% increase in survival as compared to the control. Moreover, this increase in survival was highly statistically significant (p<0.001; see statistical evaluation below).

Without being bound by theory, the biphasic nature of the dose response curve (FIG. 14) may be explained by the presence of protein aggregates in the administered IL-12. It is possible that at the 40 ng (6 μg/m²) dose, the least amount of aggregation is occurring, whereas in the middle of the dose range the protein is more aggregated, and in the higher dose range the protein is delivered as both an aggregated and non-aggregated form. Consistently, if there are more aggregates leading to some immunogenicity in the middle of the dose range, this might explain presence of a few early deaths, as compared to the control, in the middle dose range. This type of aggregation and the resultant biphasic dose response curve has been seen with other biologics.

Finally, it should be noted that the concentration actually delivered to the mice in this example may be as low as 10-20% of what is indicated. This may be caused by the loss of IL-12 protein due to adsorption on the sides of tubes and syringes used in the study, as is common with solutions of very low protein concentrations. These effects may be mitigated by various measures, including without limitation, the use of containers with reduced affinity for the non-specific protein interactions, or the pre-treatment of containers to reduce adsorption, as well as with the use of carrier molecules or proteins.

### Statistical Analysis

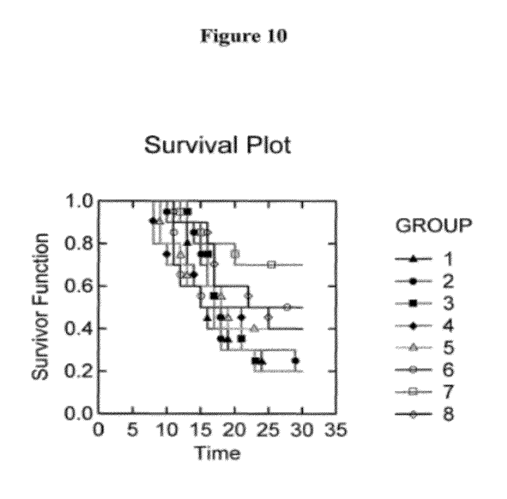

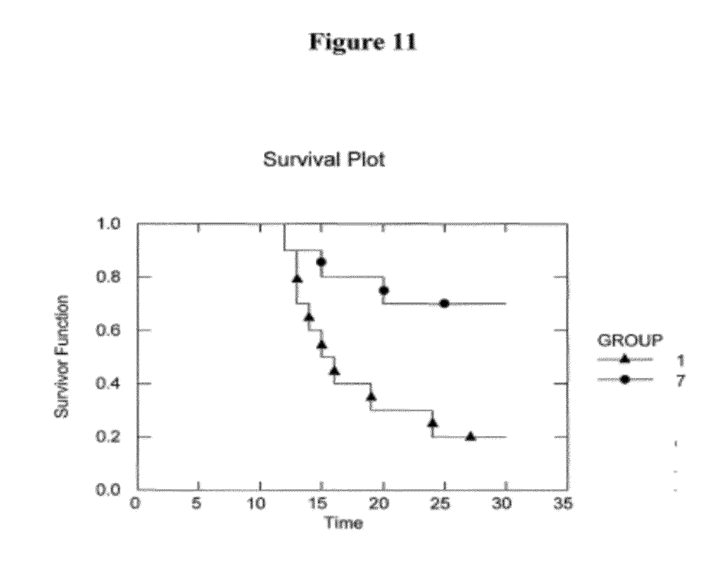

FIG. 10 shows a stratified K-M analysis of the double dose experiments. In the double dosing experiments, doses of IL-12, 200-300 ng and above, increased survival in this experiment. Notably, when group 7 (300 ng; 45 μg/m²) is compared to group 1 (0 ng, vehicle control), the effect was statistically significant (p<0.03; Tarone-Ware and Mantel methods; FIG. 11). In contrast to the single dose experiments, significant low dose therapeutic effects were not observed in the double dosing regimes. Weight loss was not a significant covariate in this experiment (FIG. 9A), suggesting that the double dose protocol allowed for considerable weight loss without death, particularly at the highest IL-12 dose.

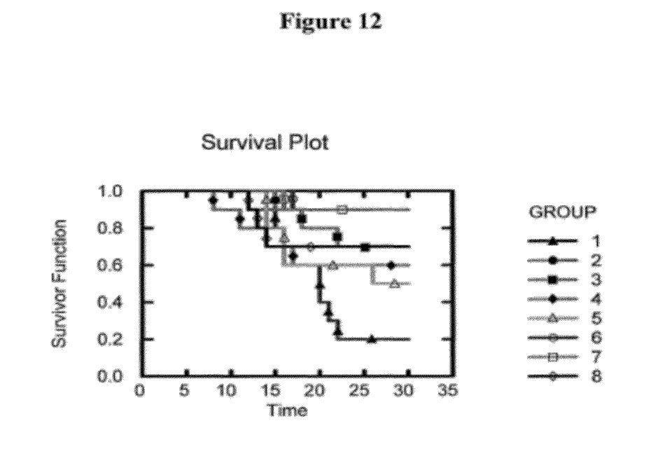

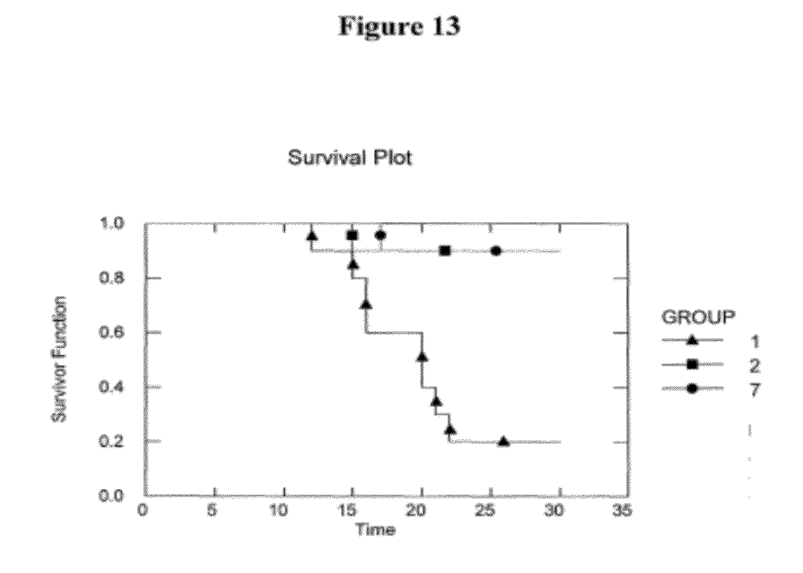

Stratified K-M analysis of the results generated in the single dose experiments indicated an overall statistically significant effect of IL-12 dose on survival time (p<0.02; Tarone-Ware method; FIG. 12). Strikingly, when groups 2 (40 ng; 6 μg/m²) and 6 (200 ng; 30 μg/m²) are compared with group 1 (0 ng, vehicle control) in a three way K-M analysis, the results are highly statistically significant (p<0.001; Tarone-Ware Method; FIG. 13).

### Example 7

### Determination of LD Values for Acute Whole Body Irradiation of C57BL/6 Female Mice

Six groups of 10 female C57BL/6 mice each were subjected to increasing doses of total body radiation using a Gammacell 40 irradiator with Cesium source. The Specific dosage activity (Adjusted Dose Rate) for the irradiator was 5159 rad/hour. Groups were irradiated for the time periods shown in Table 5 in order to achieve the final dose of radiation as delineated in the table.

TABLE 5
Experimental design for acute exposure to whole body irradiation.

| Time | Total Body Dose |
|---|---|
| (min:sec) | (Rad) |
| 9:00 | 773.85 |
| 9:20 | 802.22 |
| 9:40 | 931.45 |
| 10:00 | 859.83 |
| 10:20 | 888.21 |
| 10:40 | 917.44 |

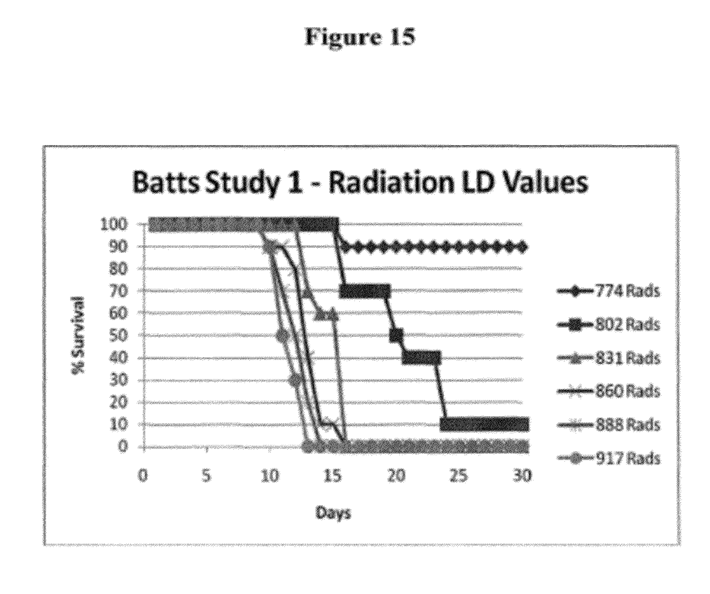

The irradiated mice were housed for 30 days following irradiation. The mice were monitored daily for survival with the day of death recorded for each mouse. The Kaplan-Meier graph of the results is presented in FIG. 15. Calculated radiation exposures for LD₃₀₍₃₀₎, LD₅₀₍₃₀₎, and LD₇₀₍₃₀₎ were about 782 rad (7.82 Gy), 788 rad (7.88 Gy), and 794 rad (7.94 Gy), respectively.

### Example 8

### Low Dose IL-12-Mediated Survival in Irradiated Mice

### Materials and Methods

At day 0, total body irradiation (TBI) was carried out at a lethal dose of 8.0 Gy (Harlan mice) or 9.0 Gy (Coats mice). These doses are expected to cause death in about 90% of animals within 30 days (LD_{90/30}). The dosage was administered using Gammacell® 40 with ¹³⁷Cs source (Theratronics, Ontario Canada), with a rate of 71 cGy/min in Coats mice studies and 85 cGy/min in the Harlan mice studies, in a specially constructed “pie-box” designed to keep mice in the center of the irradiator for even distribution of radiation.

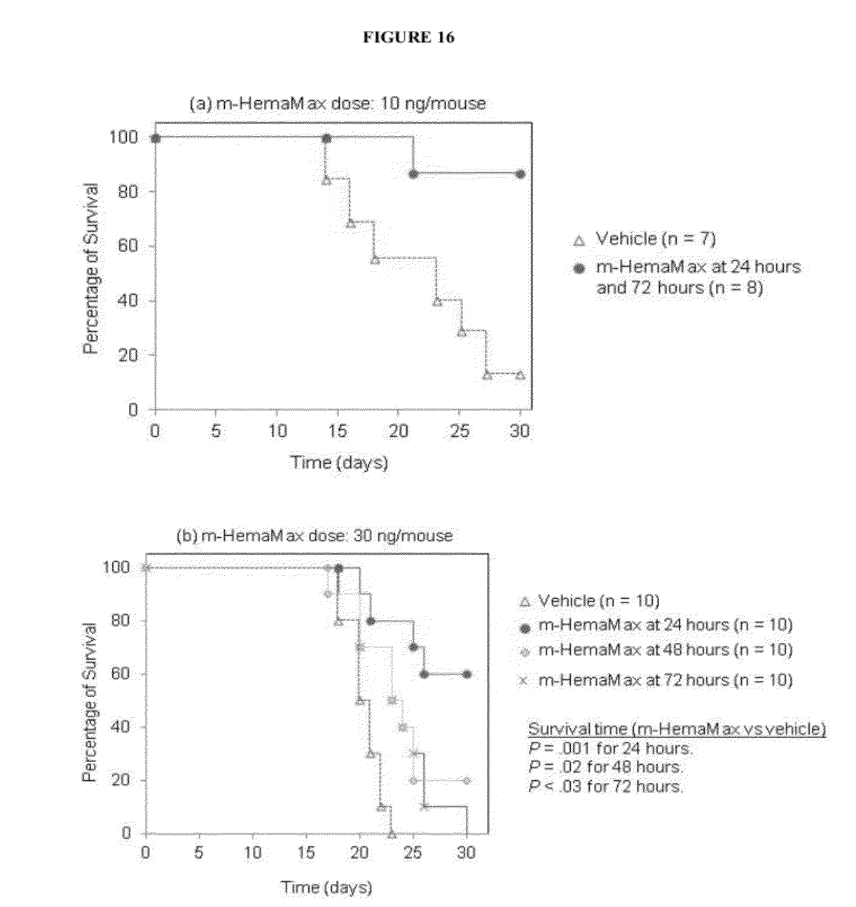

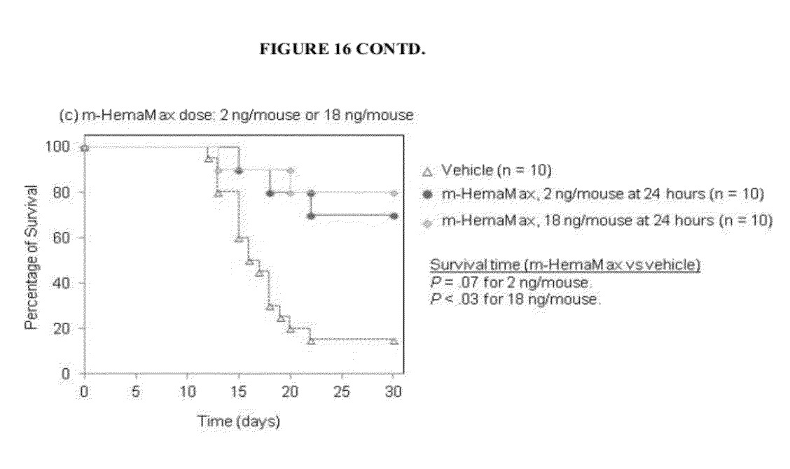

Mice received subcutaneous injections of either vehicle (PBS, pH 7.2) (n=7) or recombinant murine IL-12 (“HemaMax™” in FIG. 16) (rMuIL-12; Peprotech, Rocky Hill, N.J., USA; or SBH Sciences, Natick, Mass., USA) reconstituted in PBS at an actual dose of 10 ng/mouse at 24 hours and 72 hours after irradiation (n=8). ELISA analysis of the rMuIL-12 reconstituted in PBS demonstrated that the actual dose delivered to the mice was approximately 10% of the intended dose (100 ng/mouse), most likely because of rMuIL-12 sticking to surfaces of vials and syringes. Thus, the actual dose delivered was 10 ng/mouse, as shown in FIG. 16A.

Mice were monitored for survival up to day 30. During this period, mice were deprived of all supportive care, including antibiotics, to increase the stringency of the survival protocol. The mice had access to food and acidified water ad libitum.

### Results

As shown in FIG. 16A, 87.5% of mice receiving a subcutaneous dose of 10 ng/mouse of rMuIL-12 at 24 hours and 72 hours post TBI survived an 8 Gy for up to 30 days, whereas only 14% of vehicle mice survived lethal TBI by day 30 (P<0.005). The actual rMuIL-12 dose delivered in these studies was 10 ng/mouse.

This study was repeated using a single rMuIL-12 ostensible dose of 300 ng/mouse (actual delivered dose of 20-30 ng/mouse), as detailed in FIG. 16B. A single dose of rMuIL-12 significantly increased survival time when administered at either 24 hours (P=0.001) (n=10), 48 hours (P=0.02) (n=10), or 72 hours (P<0.03) (n=10) after a 9 Gy TBI resulting in LD_{100/30} (FIG. 16B). Mice treated with rMuIL-12 had a higher percentage of survival when rMuIL-12 was administered at 24 hours as compared to 48 hours post TBI (FIG. 16B). The difference in percentage of survival between the vehicle group (n=10) and mice treated with rMuIL-12 at 24 hours post TBI was statistically significant (0% vs. 60%, respectively; P<0.05) (FIG. 16B).

ELISA analysis of the rMuIL-12 reconstituted in PBS demonstrated that the actual dose delivered to the mice was approximately 10% of the intended dose, most likely because of rMuIL-12 sticking to surfaces of vials and syringes. Therefore, the mouse study was repeated using rMuIL-12 reconstituted in P5.6TT (trehalose; Tween 20; sodium phosphate; pH 5.6), which increased dose delivery to nearly 90% of the intended dose. With this improvement, a single rMuIL-12 dose of 2 ng/mouse or 18 ng/mouse provided significantly higher radiomitigation than did vehicle against a TBI dose of 7.9 Gy that resulted in an LD_{85/30} of TBI when administered 24 hours post radiation (FIG. 16C). At the dose of 2 ng/mouse (n=10), rMuIL-12 significantly increased percentage of survival (P<0.02) and somewhat increased survival time (P=0.07) as compared to vehicle (n=10). At the dose of 18 ng/mouse (n=10), rMuIL-12 significantly increased both the percentage of survival (P<0.005) and survival time (P<0.03) compared to vehicle. Animals treated with rMuIL-12 at a higher dose, such as 160 ng/mouse, had modestly longer survival time as compared to the vehicle group but a lower percentage of survival relative to animals treated with the 2 ng/mouse or 18 ng/mouse dose (data not shown). Thus, these findings indicate that a dose of approximately 20 ng/mouse is the optimal, efficacious dose of rMuIL-12 to increase survival.

### Example 9

### Radiation Dose Dependency of IL-12-Mediated Survival in Irradiated Mice

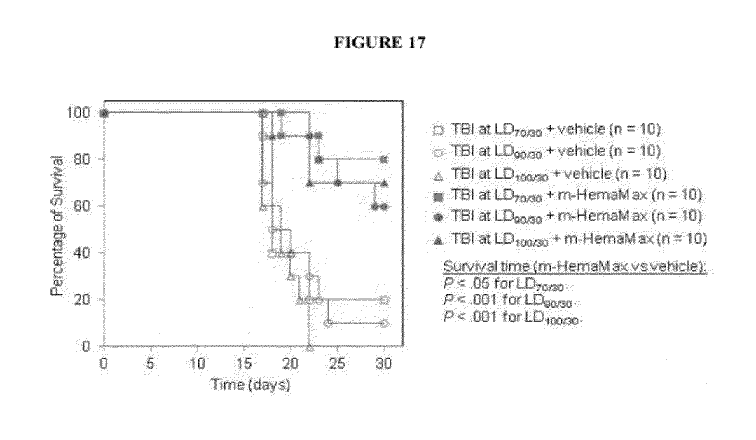

Radiation dose dependency of rMuIL-12 for survival was evaluated in mice (n=10 per group; Coats mice) irradiated at lethal doses of 8.6 Gy, 8.8 Gy, and 9.0 Gy, resulting in LD_{70/30}, LD_{90/30}, and LD_{100/30}, respectively. See FIG. 17. Animals received vehicle (P5.6TT) or rMuIL-12 (referred to as HemaMax™ in FIG. 17) (reconstituted in P5.6TT) at a dose of 20 ng/mouse 24 hours after TBI. Mice were monitored for survival up to day 30. No supportive care, including antibiotics, was allowed during this period. The mice had access to food and acidified water ad libitum.

rMuIL-12 at a dose of 20 ng/mouse administered 24 hours after TBI significantly increased survival time at all 3 levels of radiation intensities (FIG. 17). The percentage of survival in animals treated with vehicle was 20% at 8.6 Gy (LD_{70/30}) (n=10), 10% at 8.8 Gy (LD_{90/30}) (n=10), and 0% at 9.0 Gy (LD_{100/30}) (n=10) (FIG. 17). Compared to the vehicle groups, treatment with rMuIL-12 resulted in significantly higher percentage of survival of 80% at LD_{70/30} (n=10), 60% at LD_{90/30} (n=10), and 70% at LD_{100/30} (n=10) (P<0.05 for all) (FIG. 17), demonstrating a radiation dose-independence for rMuIL-12 administration at 24 hours post TBI within the selected window of radiation exposures. Remarkably, comparable percentages of survival after a single, fixed dose of rMuIL-12 at increasing radiation doses indicate that the efficacy of rMuIL-12 is not decreased with increasing radiation dose.

These data suggest that at radiation doses where immune, bone marrow, and GI damage overlap, rMuIL-12 provides mitigation of injury in all three radiosensitive tissues, thereby leading to an increase in survival that is independent of radiation dose within a certain window of exposure.

### Example 10

### Plasma PK and PD of rMuIL-12 and IFN-γ in Irradiated and Non-Irradiated Mice

### Materials and Methods

A study was done to determine the effect of irradiation on plasma concentrations of rMuIL-12 and IFN-γ. Mice (n=3 per group) received rMuIL-12 subcutaneously at a dose of 10 ng/mouse (FIG. 18A), 20 ng/mouse (FIG. 18B), 40 ng/mouse (FIG. 18C), or 200 ng/mouse (FIG. 18D) either in the absence of irradiation or 24 hours after an LD_{100/30} (8.6 Gy; Charles River mice) of TBI. Two additional control groups of animals (n=3 per group), which did not receive rMuIL-12 (referred to as HemaMax™ in FIG. 18), were either not exposed to radiation or irradiated at 8.6 Gy. The concentrations of rMuIL-12 and IFN-γ were determined in plasma from blood samples withdrawn at 45 minutes and 1.5, 3, 6, 12, 24, 48 and 72 hours after rMuIL-12 administration by enzyme-linked immunosorbent assay (ELISA). See FIG. 18

Blood samples from mice were collected into tubes containing ethylenediaminetetraacetic acid and were kept on ice (<30 minutes) until centrifugation. Samples were centrifuged at 1500×g for 10 minutes at 4° C. Plasma was aliquoted and stored at −70° C. until use. Plasma rMuIL-12, and its potential biomarkers were assayed by ELISA. The ELISA kits for mouse IL-12 (p70) and IFN-γ were obtained from BioLegend (San Diego, Calif., USA). All assays were carried out in triplicate according to the manufacturers instructions.

### Results

The exposure to rMuIL-12 (area under the curve last; AUCₗₐₛₜ) increased dose proportionally from 10 ng/mouse to 40 ng/mouse, regardless of the presence or absence of irradiation (FIGS. 18A-C and Table 6). Interestingly, maximum plasma concentrations (Cₘₐₓ) of rMuIL-12 were consistently higher in irradiated mice as compared to non-irradiated mice at all doses (FIGS. 18A-D). The exposure to rMuIL-12 (AUCₗₐₛₜ) at the dose of 200 ng/mouse was disproportionately higher than those at the lower doses (10 ng/mouse to 40 ng/mouse), suggesting that PK properties of rMuIL-12 are non-linear at the higher dose ranges (FIG. 18D; Table 6). In the dose range of 10 ng/mouse to 40 ng/mouse (FIGS. 18A-C), rMuIL-12 reached Cₘₐₓ in 3 hours to 6 hours and was eliminated with a half-life of approximately 4 hours (Table 6).

TABLE 6
Plasma PK Characteristics of m-HemaMax
in Irradiated and Non-Irradiated Mice.

| m-HemaMax | Cₘₐₓ | | AUCₗₐₛₜ | | Tₘₐₓ | | t_{1/2} | |
|---|---|---|---|---|---|---|---|---|
| dose, | (pg/mL) | | (pg · h/mL) | | (hours) | | (hours) | |
| ng/mouse | NR | R | NR | R | NR | R | NR | R |
| 10 | 82.8 | 96.4 | 628 | 728 | 6 | 3 | na | na |
| 20 | 129.5 | 217.2 | 1453 | 2364 | 3 | 6 | 3.7 | 3.5 |
| 40 | 257.8 | 308.8 | 2720 | 2701 | 6 | 3 | 3.5 | 4.2 |
| 200 | 1428 | 2332 | 21008 | 37059 | 3 | 1.5 | 4.8 | 7.2 |

rMuIL-12 administration increased plasma IFN-γ concentration with a lag time at all study doses (FIGS. 18A-D). Of significance, IFN-γ production was not abrogated in irradiated mice (FIGS. 18A-D). In fact, for all rMuIL-12 doses, except the optimal dose of 20 ng/mouse dose, plasma IFN-γ levels were higher in irradiated mice compared to non-irradiated mice (FIGS. 18A, C, and D). The exposure to IFN-γ dose proportionally increased as a function of increasing rMuIL-12 dose from 10 ng/mouse to 200 ng/mouse (data not shown). IFN-γ was not detected in plasma of mice which did not receive rMuIL-12 regardless of the presence or absence of irradiation.

### Example 11

### Plasma PK and PD of EPO in Irradiated and Non-Irradiated Mice

To assess whether to rMuIL-12 affects plasma levels of erythropoietin (EPO) in irradiated and non-irradiated mice, plasma EPO was measured in samples from the study described in Example 10. Plasma EPO was measured at one early timepoint, 12 hours after rMuIL-12 administration (FIG. 19).

In non-irradiated, untreated animals, EPO was detectable in plasma at low pg/mL range (FIG. 19). Irradiation increased plasma EPO levels nearly linearly up to 80 hours post TBI, suggesting that EPO is a part of the physiological response to radiation injury (data not shown). At the optimal dose of 20 ng/mouse at 12 hours post-administration (36 hours post radiation exposure), rMuIL-12 (HemaMax™ in FIG. 19) substantially increased plasma EPO concentrations over the radiation-induced levels (FIG. 19), indicating that rMuIL-12 potentiates the EPO-mediated physiological response to radiation, but only at or near the optimal dosing level.

At this optimal dose, plasma EPO levels were also increased in non-irradiated mice (FIG. 19). The IFN-γ response appeared to be subdued at the 20 ng/mouse dose of rMuIL-12, the dose at which EPO was upregulated by rMuIL-12, as compared to the other doses assessed. In a mouse model of multiple sclerosis, administration of EPO was reported to downregulate the inflammatory response, and in particular, suppress IFN-γ (Melichar et al., J. Immunother., 26: 270-276 (2003)).

Thus, these findings show that increased plasma EPO levels may play a role in the suppression of plasma IFN-γ levels in irradiated mice that received to rMuIL-12 at the dose of 20 ng/mouse (see FIG. 18B), leading to a decrease in the inflammatory response to radiation.

Other biomarkers of rMuIL-12 administration were also screened, namely tumor necrosis factor-alpha (TNF-α) and stem cell factor (SCF), but plasma levels for these factors were found to be below the limit of quantitation.

### Example 12

### Effects of rMuIL-12 Administration in Mouse Bone Marrow and Small Intestine

### Materials and Methods

For bone marrow histopathology studies, mice (n=2 per group) were subjected to TBI at 8.0 Gy (Harlan mice, ˜LD_{40/30} in this experiment) and were subsequently administered either vehicle (P5.6TT) or rMuIL-12 (20 ng/mouse) subcutaneously at either (a) 24 hours, (b) 24 hours and 2 days, (c) 24 hours and 3 days, (d) 24 hours and 4 days, or (e) 24 hours and 5 days after irradiation. An additional group of mice (n=2) received rMuIL-12 at 24 hours after TBI. Mice were sacrificed 12 days after irradiation, and femoral bone marrow was provided as paraffin-embedded, sectioned tissues by Cyto-Pathology Diagnostic Center, Inc (Duarte, Calif., USA).

For GI histopathology studies, mice (n=3 per group) received vehicle (P5.6TT) or rMuIL-12 subcutaneously at doses from 10 ng/mouse to 200 ng/mouse either in the absence of irradiation or 24 hours after a TBI at 8.6 Gy (Charles River mice, LD_{100/30}). Mice were sacrificed 3 days after irradiation, and jejunum was provided as paraffin-embedded, sectioned tissues by Cytopathology Diagnostics Center, Inc. (Duarte, Calif., USA).

Sectioned tissues were deparaffinized with xylene, rehydrated with decreasing concentrations of ethanol, and subjected to the heat-induced epitope retrieval (HIER) to recover antigens. Endogenous peroxidase was inhibited with 0.3% H₂O₂, and background staining was blocked with the Background Sniper (Biocare Medical, LLC.; Concord, Calif.).

In the bone marrow histopathology studies, tissue sections were incubated with either rabbit anti-mouse IL-12 receptor beta 2 subunit (IL-12Rβ2) (Sigma; St Louis, Mo.), rabbit anti-mouse osteocalcin (Millipore; Billerica, Mass.), a marker of osteoblasts, or rabbit anti-mouse Sca-1 (Epitomics; Burlingame, Calif.), a marker of hematopoietic stem cells. In the GI histopathology studies, tissue sections were incubated with rabbit anti-mouse IL-12Rβ2, or rabbit anti-mouse leucine-rich-repeat-containing G-protein-coupled receptor 5 (LGR5), a GI stem cell marker that is expressed upon GI injury. After removing the primary antibodies, tissue sections were incubated with peroxidase conjugated anti-rabbit IgG (ImmPRESS; Vector Laboratories; Burlingame, Calif.). Red coloring of peroxidase labeled cells developed following incubation with AEC substrate (ImmPACT AEC; Vector Laboratories; Burlingame, Calif.) and were counterstained with CAT Hematoxylin (Biocare Medical, Concord, Calif.). Tissue sections were then immersed in Vectamount (Vector Laboratories; Burlingame, Calif.), covered with a cover slip, sealed with clear nail polish, and visualized using an Olympus Compound microscope (Olympus America, Inc; Center Valley, Pa.) at 100× magnification for bone marrow sections and 400× for jejunum.

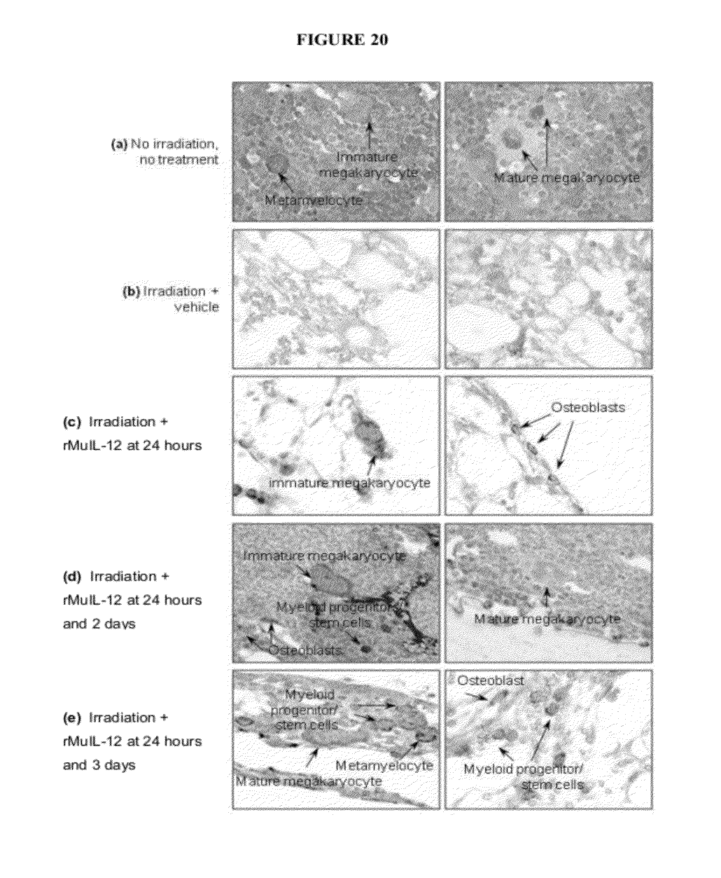

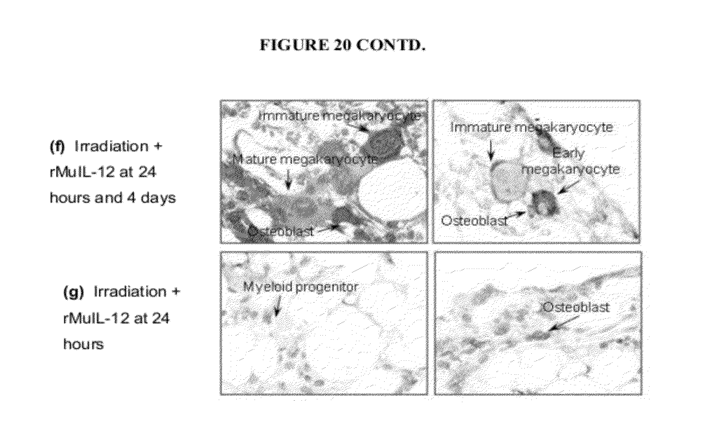

Co-expression of Sca-1 and IL-12Rβ2 on hematopoietic stem cells was evaluated by incubating bone marrow tissue sections first with rabbit anti-mouse Sca-1 (Epitomics, Burlingame, Calif.) followed by incubation with Rabbit on Rodent HRP-Polymer (Biocare Medical; Concord, Calif.) and 3,3′-diaminodbenzidine substrate (Biocare Medical; Concord, Calif.). After treatment with denaturing solution (Biocare Medical; Concord, Calif.), tissue sections were incubated with rabbit anti-mouse IL-12Rβ2 (Sigma; St Louis, Mo.) followed by incubation with Rabbit on Rodent AP polymer (Biocare Medical; Concord, Calif.) and Warp Red substrate (Biocare Medical; Concord, Calif.). Tissue sections were then counterstained in CAT Hematoxylin and visualized as described above. Using this method, cells expressing Sca-1 and IL-12Rβ2 were stained in brown and pink, respectively. IL-12Rβ2—expressing hematopoietic stem cells were identified by co-staining for Sca-1 (a murine stem cell marker; see below), immature megakaryocytes exhibited lobulated nuclei surrounded by a narrow rim of cytoplasm, matured megakaryocytes exhibited lobulated nuclei and voluminous cytoplasm, and myeloid progenitor cells in the metamyelocyte stage (FIG. 20A).

### Results

Bone marrow from non-irradiated, untreated mice was characterized with the presence of IL-12Rβ2—expressing hematopoietic stem cells, identified by co-staining for Sca-1 (a murine stem cell marker; see below), immature megakaryocytes with lobulated nuclei surrounded by a narrow rim of cytoplasm, matured megakaryocytes with lobulated nuclei and voluminous cytoplasm, and myeloid progenitor cells in the metamyelocyte stage (FIG. 20A). Bone marrow from mice treated only with vehicle and subjected to an LD_{30/30} of TBI (8.0 Gy) was characterized with minimal signs of hematopoietic regeneration and the complete lack of IL-12Rβ2—expressing cells after 12 days following irradiation (FIG. 20B). In contrast, mice treated with various dosing regimens of rMuIL-12 showed varying levels of hematopoietic reconstitution, which was characterized with the presence of IL-12Rβ2—expressing myeloid progenitors, megakaryocytes, and osteoblasts (FIGS. 20C-F). Mice treated with rMuIL-12, which has been demonstrated to not cross react with the murine IL-12 receptor, showed some signs of regeneration, however, lacked megakaryocytes (FIG. 20G). For mice treated with rMuIL-12, however, no increase in the survival was observed, as compared with the vehicle control group (data not shown).

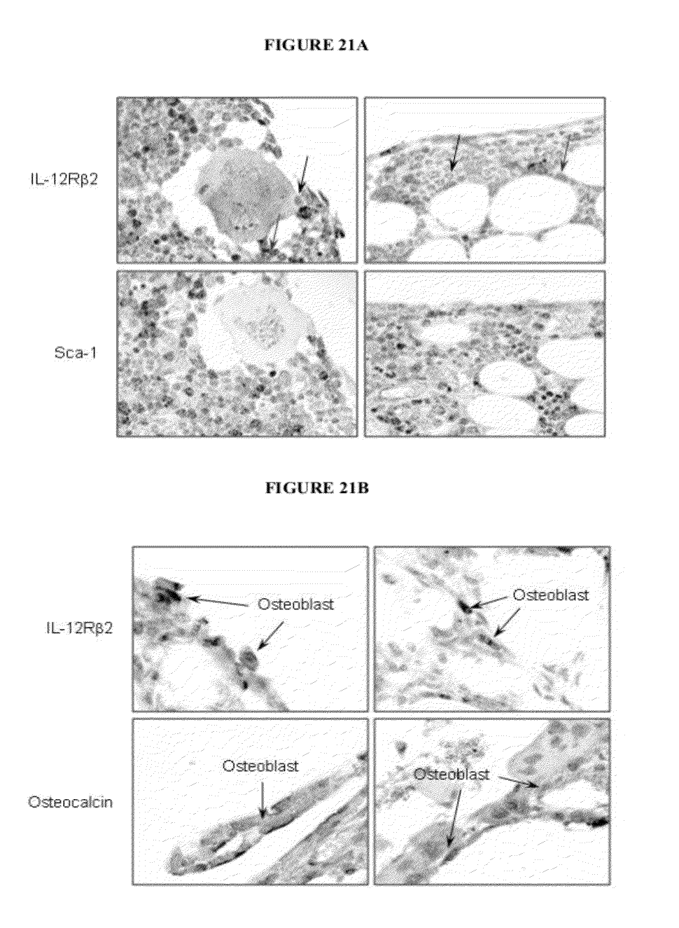

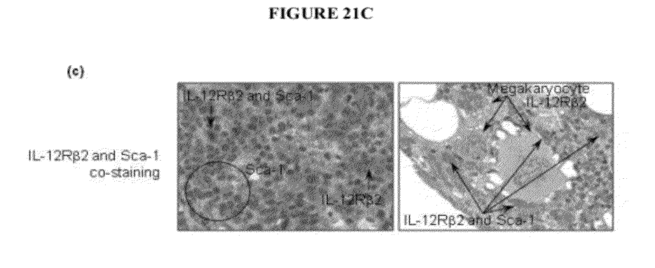

To further evaluate whether morphologically identified cells were indeed hematopoietic stem cells and osteoblasts, bone marrow tissue sections were stained for the corresponding markers, respectively, Sca-1 and osteocalcin. As depicted in FIGS. 21A and 21B, IL-12Rβ2 expression was observed on cells that were morphologically identified as hematopoietic stem cells and osteoblasts and expressed Sca-1 and osteocalcin, respectively. As depicted in FIG. 21c, a discrete subset of hematopoietic stem cells were co-stained for the presence of both IL-12Rβ2 and Sca-1. Both immature and mature megakaryocytes expressing IL-12Rβ2 was also evident in the bone marrow tissue sections (FIG. 21C). These findings show a direct role for IL-12 signaling pathway in hematopoietic reconstitution.

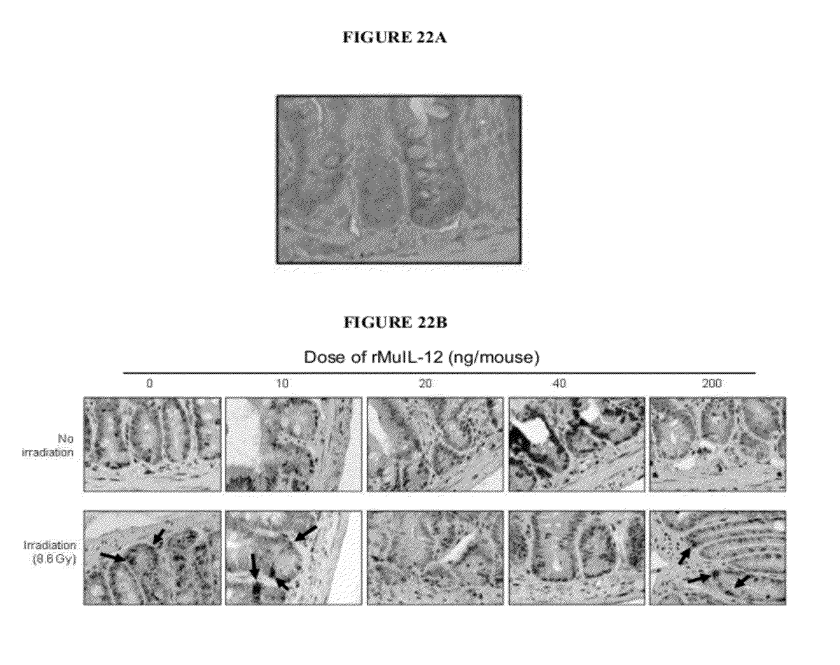

Similar to hematopoietic stem cells and osteoblasts in femoral bone marrow, mice jejunal crypts expressed IL-12Rβ2 (FIG. 22A). In the absence of irradiation, rMuIL-12 administration at doses up to 200 ng/mouse did not cause injury in jejunal crypts (FIG. 22B, upper panel). Exposure to TBI (8.6 Gy), however, resulted in substantial jejunal damage 3 days after irradiation, as evidenced by the widespread expression of LGR5, a GI stem cell marker shown to be expressed upon chemotherapy-induced GI injury.

Administration of rMuIL-12 at the low dose range of 10 ng/mouse to 40 ng/mouse dose-dependently mitigated radiation-induced jejunal damage, with no LGR5 expression evident at the optimal efficacious dose of 20 ng/mouse (FIG. 22B, lower panel). However, rMuIL-12 at the high dose of 200 ng/mouse exacerbated jejunal injury (FIG. 22B, lower panel).

As observed with the rMuIL-12 dose ranges for optimal increases in survival, these data reveal a window of opportunity for mitigation of radiation injury by rMuIL-12 in a very low dose range that is also effective in alleviating bone marrow damage.

### Example 13

### IL-12 Dose Equivalency Between Murine and Primate PBMC

To achieve a similar radiomitigation effect in rhesus monkey, doses that are pharmacologically equivalent to those given to mice should be administered to rhesus monkeys. Based on the Food and Drug Administration (FDA) guidelines, the optimal 20 ng/mouse dose (1000 ng/Kg) and a non-optimal 80 ng/mouse (4000 ng/Kg) dose in mouse translate, respectively, to doses of 250 ng/Kg and 1000 ng/Kg in rhesus monkey. However, eliciting a pharmacologically equivalent response at species-specific equivalent doses depends on several factors including similar drug exposure and specific reactivity with the primary target site in both species. Therefore, prior to evaluating the efficacy of the radiomitigation effects of recombinant IL-12 in non-human primates (NHP), the pharmacological equivalency of the species-specific equivalent doses was examined in CD14-negative peripheral blood mononuclear cells (PBMC) from mouse, rhesus monkey, and human.

### Materials and Methods

Human PBMC collected by apheresis were purchased from AllCells (Emeryville, Calif., USA). Mouse and rhesus monkey PBMC were from Bioreclamation (Liverpool, N.Y., USA). CD14-PBMC were isolated as follows. Red blood cells were removed from human PBMC by a single step gradient with Ficoll-Hypaque premium (Density=1.077; GE Healthcare Lifesciences; Piscataway, N.J., USA) and from rhesus monkey and mouse PBMC by lysis using ACK lysis buffer (Invitrogen; Carlsbad, Calif., USA). To remove IL-12-secreting endogenous monocyte populations, human and rhesus monkey PBMCs were labeled with mouse anti-human CD14PE antibody (AbD Serotec; Raleigh, N.C., USA), and mouse PBMCs were labeled with mouse anti-mouse CD14PE (AbD Serotec; Raleigh, N.C., USA). The excess antibody was removed and cells were incubated with magnetic beads conjugated with anti-PE antibody (Miltenyi Biotec; Auburn, Calif., USA). After removing the excess antibody, CD14⁺ cells were captured by adsorption to an LD column (Miltenyi Biotec; Auburn, Calif., USA) immobilized in a magnetic field (Quadro MACS®; Miltenyi Biotec; Auburn, Calif., USA). CD14⁻ cells in the flow-through were collected, and those from humans were resuspended at a density of 14×10⁶ cells/mL in cold fetal bovine serum (FBS) containing 20% dimethyl sulfoxide whereas those from rhesus monkey and mouse were resuspended at a density of 2.14×10⁶ cells/mL in RPMI medium containing 10% FBS and antibiotics.

IFN-γ was quantified by ELISA in supernatants from 2.5×10⁵ human, rhesus monkey, or mouse CD14⁻ PBMC incubated with various concentrations (range: 0 to 1000 pM) of recombinant human IL-12 (rHuIL-12) or recombinant murine IL-12 (rMuIL-12) for 16 hours at 37° C. All experiments were carried out in triplicate. The half maximal effective concentration (EC₅₀) of IL-12 for stimulating IFN-γ secretion was calculated by SoftMax Pro® software version 3.1 (Molecular Devices; Sunnyvale, Calif., USA) using a 4-parameter logistic fit.

### Results

Target reactivity to rHuIL-12 was evaluated by comparing EC₅₀ values of rHuIL-12 and rMuIL-12 for stimulating the secretion of IFN-γ from CD14⁻ PBMC. rHuIL-12 did not cross-react with PBMC isolated from mouse and rat (EC₅₀>1000 pM). In contrast, rHuIL-12 and rMuIL-12 potently stimulated IFN-γ secretion from both human and rhesus monkey PBMC with EC₅₀ values of, respectively, 2.51±0.51 pM and 1.05±0.10 pM. The EC₅₀ value of rMuIL-12 for stimulating IFN-γ secretion from mouse PBMC was 0.35±0.29 pM. These findings show that the reactivities of monkey and mouse PBMC to, respectively, rHuIL-12 and rMuIL-12 are similar in relation to IFN-γ secretion in vitro.

### Example 14

### Plasma Pharmacokinetics of rHuIL-12 in Rhesus Monkeys

### Materials and Methods

Radiation-naive male rhesus monkeys received rHuIL-12 subcutaneously at a dose of either 250 ng/Kg (n=3) or 1000 ng/Kg (n=3). The concentrations of rHuIL-12, IFN-γ, and other potential biomarkers of rHuIL-12 were determined by ELISA (as described in Example 15) in plasma samples withdrawn prior to the rHuIL-12 administration and at 2, 6, 12, 18, 24, 30, 36, 48, 72, 96, 120, 144 and 168 hours after rHuIL-12 administration.

### Results

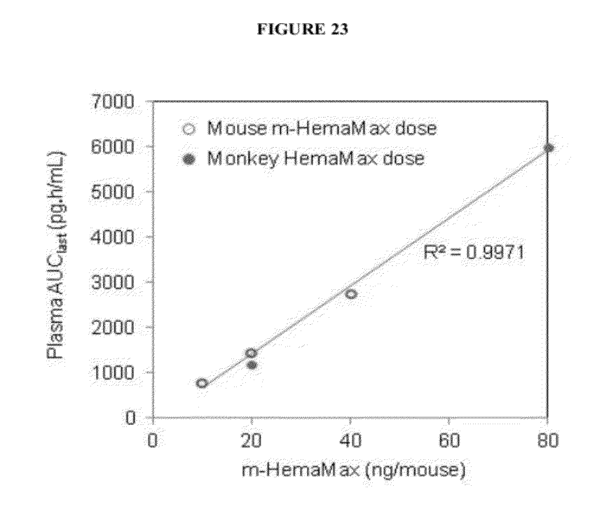

Plasma pharmacokinetics (PK) of rHuIL-12 was examined in rhesus monkeys following a single administration of rHuIL-12 at two doses of 250 ng/Kg and 1000 ng/Kg in the absence of irradiation. Following administration, the exposure (AUCₗₐₛₜ) to rHuIL-12 increased in proportion to dose (Table 7). The AUCₗₐₛₜ of rHuIL-12 in rhesus monkey was perfectly superimposed linearly for the AUCₗₐₛₜ of rMuIL-12 in mice over the dose range of 10 ng/mouse to 80 ng/mouse (FIG. 23), suggesting that the species-specific equivalent doses calculated from mice studies provided similar drug exposure in monkeys. The 200 ng/mouse dose was not included in this analysis as it appeared that rMuIL-12 exhibits different PK characteristics at higher doses (Table 6).

TABLE 7
Plasma PK Characteristics of HemaMax in Non-Irradiated
Rhesus Monkeys.

| HemaMax dose, | Cₘₐₓ | AUCₗₐₛₜ | Tₘₐₓ | t_{1/2} |
|---|---|---|---|---|
| ng/Kg | (pg/mL) | (pg · h/mL) | (hours) | (hours) |
| 250 | 38.3 ± 8.4 | 1192 ± 382 | 10 ± 3.5 | 20.4 ± 12.3 |
| 1000 | 193.3 ± 61.3 | 5708 ± 1488 | 8 ± 3.5 | 40.6 ± 24.1 |

rHuIL-12 at a single dose of 250 ng/Kg or 1000 ng/Kg was well tolerated and was not associated with overt signs of toxicity, except for occurrences of transient decreases in appetite in the 1000 ng/Kg group.

### Example 15

### Biomarker Expression Following Administration of rhuIL-12 in Rhesus Monkeys

### Materials and Methods

Blood samples from rhesus monkeys treated as described in Example 14 were collected into tubes containing ethylenediaminetetraacetic acid and were kept on ice (<30 minutes) until centrifugation. Samples were centrifuged at 1500×g for 10 minutes at 4° C. Plasma was aliquoted and stored at −70° C. until use. Plasma rHuIL-12 and its potential biomarkers were assayed by ELISA.

The ELISA kits for non-human primate (NHP) IL-12 were obtained from BioLegend (San Diego, Calif., USA), MabTech (Mariemont, Ohio, USA), and R&D Systems (Minneapolis, Minn., USA). ELISA kits for NHP IFN-γ were obtained from MabTech (Mariemont, Ohio, USA), for human EPO, IL-18, and IL-15 from R&D Systems (Minneapolis, Minn., USA), and for neopterin from GenWay (San Diego, Calif., USA). All assays were carried out in triplicate according to the manufacturers' instructions except those for NHP IL-12 in which an in-house reference standard was used instead of the standard provided by the manufacturer.

### Results

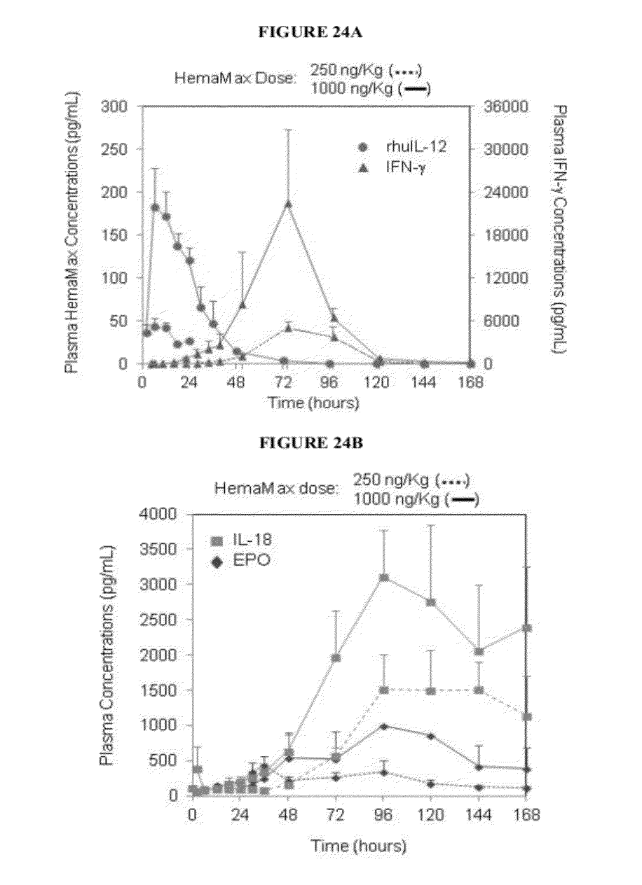

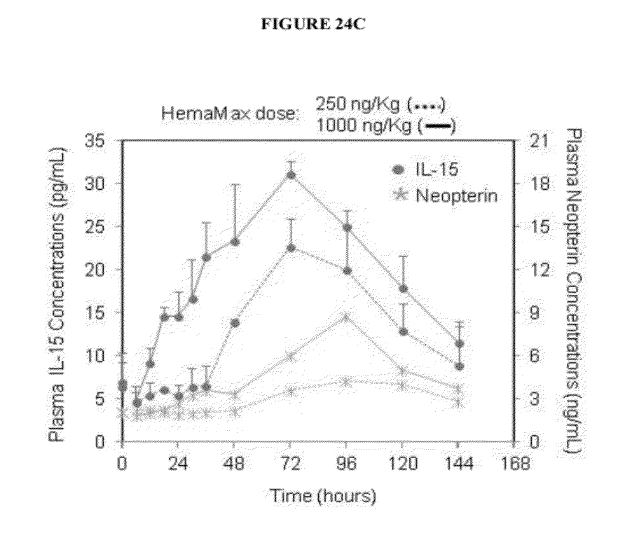

In monkeys, subcutaneous administration of rHuIL-12 appeared in plasma shortly after administration and was not detectable after 72 hours (FIG. 24A). Moreover, as observed in mice with rMuIL-12, rHuIL-12 was observed to increase plasma IFN-γ concentration in proportion to dose (FIG. 24A). Temporal kinetics of IFN-γ response in rhesus monkey was, however, different from mouse in that the IFN-γ response was delayed for a longer period of time and was much higher in magnitude (FIG. 24A). Neither rHuIL-12 nor IFN-γ was detected in plasma of monkeys that did not receive rHuIL-12.

Of other potential biomarkers, the exposure (AUCₗₐₛₜ) to IL-18 and EPO was increased by 2.4-fold and 5.1-fold, respectively, as the rHuIL-12 dose was increased from 250 ng/Kg to 1000 ng/Kg (FIG. 24B). rHuIL-12 also increased plasma IL-15 and neopterin concentrations, peaking at 72 hours and 96 hours, respectively, post rHuIL-12 administration (FIG. 24C). The plasma concentrations of rhesus monkey TNF-α and IL-10 were not changed. Numerous other potential biomarkers were screened, but were found to be below the limit of quantification.

### Example 16

### Expression of IL-12Rβ2 in Bone Marrow and Small Intestine of Monkeys and Humans

### Materials and Methods

Paraffin-embedded, sectioned tissues from non-human primates (NHP) and human femoral bone marrow and jejunum/ileum were obtained from Biomax, Inc (Rockville, Md.). NHP and human tissue sections were immunohistochemically stained IL-12Rβ2 using rabbit anti-human IL-12Rβ2 according to the procedures described in Example 12.

### Results

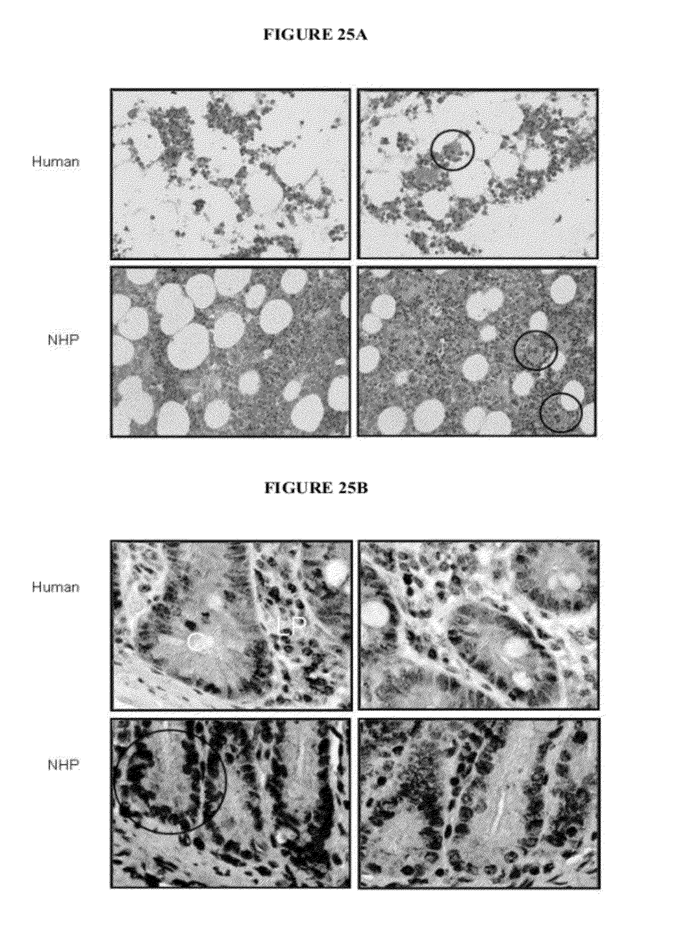

The expression of IL-12Rβ2 in non-irradiated non-human primates (NHP; rhesus monkeys) and human femoral bone marrow and jejunum/ileum was evaluated by immunohistochemistry. As depicted in FIG. 25A, NHP, as well as human, progenitor cells and megakaryocytes expressed IL-12Rβ2. The expression of IL-12Rβ2 was also found on osteoblasts/osteoclasts from the bone marrow. Bone marrow adipocytes were not stained positive for IL-12Rβ2.

In the small intestine, IL-12Rβ2 was most commonly expressed in crypts (FIG. 25B). IL-12Rβ2 expression was also noted in lymphoid cells populating the lamina propria and submucosal regions (FIG. 25B). Mucin secreting goblet cells did not express IL-12Rβ2. Both crypt and lamina propria IL-12Rβ2-expressing cells could represent multifunctional mesenchymal-origin myofibroblasts that can serve as crypt shape-forming cells that also occupy both a stem cell niche and act as non-professional antigen presenting cells to immunomodulatory cells in the lamina propria.

### Example 17

### rHuIL-12 Administration in Irradiated Rhesus Monkeys

### Materials and Methods

At day 0, rhesus monkeys were subjected to TBI at an LD_{50/30} of 6.7 Gy. Irradiation was performed in two half-dose fractions (anteroposterior and posteroanterior) at the rate of 55 cGy/minute using a Cobalt-60 unit (Theratron 780; Theratronics; Ontario, Canada). The irradiation dose was monitored with 2 dosimeters (Thermoluminescent or NanoDot dosimeters; Landauer Inc.; Glenwood, Ill., USA) placed at the apex of the sternum and at the corresponding level in the interscapular area of each animal. Following TBI, animals were randomly assigned to receive subcutaneously either (a) vehicle (P5.6TT) at 24 hours post TBI (n=8), (b) 100 ng/Kg of rHuIL-12 at 24 hours post TBI (n=8), (c) 100 ng/Kg of rHuIL-12 at 24 hours and 7 days post TBI (n=8), (d) 250 ng/Kg of rHuIL-12 at 24 hours post TBI (n=8), or (e) 250 ng/Kg of rHuIL-12 at 24 hours and 7 days post TBI (n=8). Animals were monitored for survival and clinical and physical characteristics for up to day 30. The primary outcome measure was the percentage of survival. Peripheral blood cell counts, body weight, and clinical signs were evaluated as secondary outcome measures. During the study, blood transfusions or antibiotic use was prohibited.

### Results

In a study of 40 animals, the percent survival of rhesus monkeys exposed to an LD_{50/30} of TBI (6.7 Gy) was determined following treatment with 100 ng/Kg or 250 ng/Kg of rHuIL-12 administered at 24 hours or at 24 hours and 7 days post TBI. This study was conducted in the absence of any supportive care, including antibiotics. The doses of rHuIL-12 were chosen based on PK/PD studies in rhesus monkeys and were equivalent to rMuIL-12 doses of 8 ng/mouse and 20 ng/mouse, respectively.

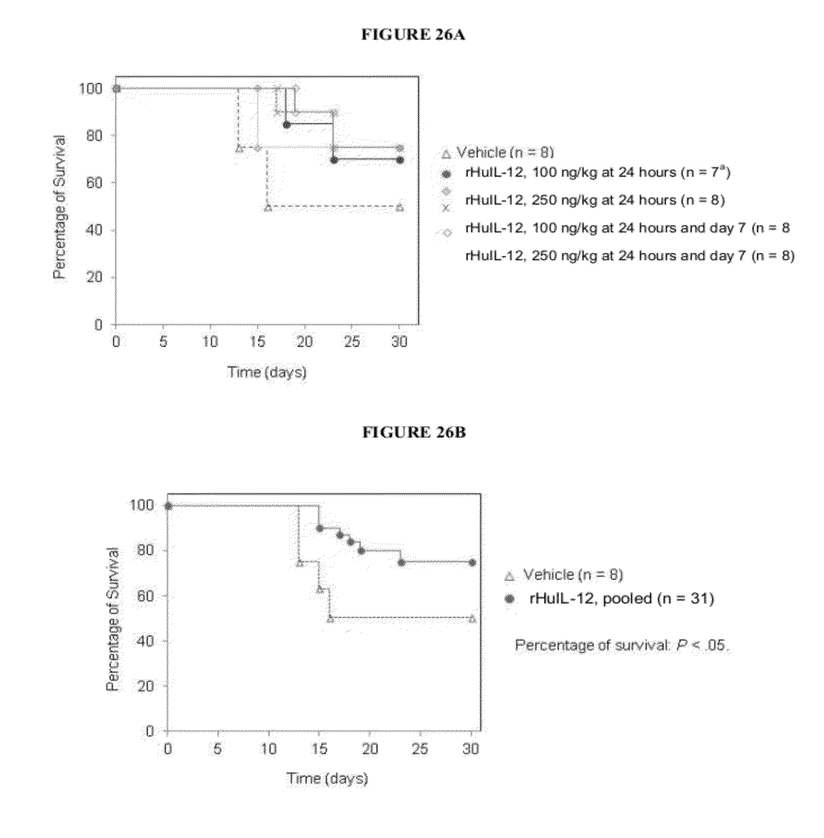

As depicted in FIG. 26A, rHuIL-12 at both doses, following either single or two administrations, mitigated death due to irradiation to the same extent. Overall percentages of survival were 71% in the 100 ng/Kg single dose group (n=7) and 75% in all other groups receiving rHuIL-12 (n=8) compared to 50% in the vehicle group. This is a dramatic and significant 21-25% improvement in survival. Between-group differences in percentage of survival were not statistically significant, most likely because of the small number of animals in each group (n=8), but also because both rHuIL-12 doses were likely within the efficacious dose range. However, analysis of the percent survival regardless of the rHuIL-12 dosing regimen indicated that when pooled together, monkeys treated with rHuIL-12 had significantly higher percent survival than those receiving vehicle (75% vs. 50%, respectively; P=0.05) (FIG. 26B), demonstrating at 25% increase in survival with administration of rHuIL-12.

### Example 18

### Blood Cell Counts in Irradiated Rhesus Monkeys Treated with rHuIL-12

### Materials and Methods

Blood cell counts of leukocytes and platelets were analyzed in samples from monkeys treated as described in Example 17.

### Results

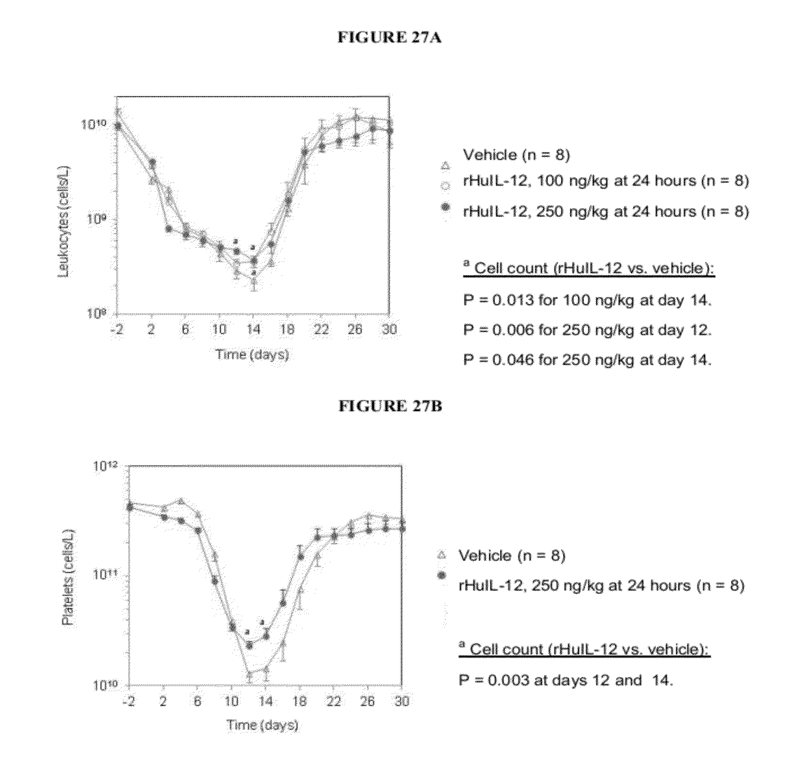

Three analyses were conducted to assess differences in blood cell counts during the study period. In the first analysis, where blood cell counts were analyzed from day 1 up to day 30, animals treated with rHuIL-12 had significantly higher numbers of leukocytes and thrombocytes at days 12 and 14, around the nadir, for the 100 ng/Kg and 250 ng/Kg doses, as compared to animals treated with vehicle (FIGS. 27A-B).

In a second analysis, in which blood cell counts were analyzed from day 1 up to day 14, the day before any animals died, animals treated with rHuIL-12 had higher platelets counts compared to animals treated with vehicle (P=0.079 for the 250 ng/Kg group and P=0.02 for the 100 ng/Kg twice dosing group) during nadir (days 12 to 14). Additionally, in comparison to the vehicle group, animals treated with rHuIL-12 had significantly higher counts of leukocytes (P<0.01 for the 250 ng/Kg group and P<0.04 for the 100 ng/Kg twice dosing group) and reticulocytes (P<0.04 for the 250 ng/Kg group and P<0.001 for the 100 ng/Kg group) during nadir (days 12 to 14). The same trend was apparent for neutrophil, basophil, and lymphocyte counts, but they did not reach acceptable levels of statistical significance.

In a third analysis, the number of animals that reached clinically low platelet counts during the study was assessed. This analysis revealed a remarkable difference between the vehicle and rHuIL-12 groups in the number of platelet counts dropping below a threshold level of 20,000 platelets/μL, a level generally necessitating platelet transfusion. In the rHuIL-12 250 ng/Kg group, only 4 out of 16 (25%) platelet counts at the nadir (day 12 to day 14) dropped below the transfusion threshold of less than 20,000 platelets/μL, whereas 12 out of 15 (80%) platelet counts for the vehicle animals were below the threshold level during the same period of time (P=0.007).

Taken altogether, these findings show that rHuIL-12 increases leukocytes, platelet, and reticulocyte counts just prior to the days on which animals begin to die from radiation toxicity (see FIG. 26A, day 13). Vehicle-treated animals that survived up to day 30 also had quick recovery of blood cell counts, which were statistically indistinguishable from those in the rHuIL-12 groups. These findings demonstrate that mortality likely occurs in animals that do not show a strong blood cell recovery around the nadir day(s). The validity of this hypothesis was evaluated by comparing blood cell counts of animals stratified by the mortality status, i.e., those surviving up to day 30 versus animals dying after day 12. In this analysis, the blood cell counts on the day before death was taken for animals that died after day 12. The comparison day for the surviving animals in each group was the average day on which the decedents in a particular group died (days 14 to 18).

This analysis demonstrates that, regardless of the particular treatment group, animals surviving up to day 30 had significantly higher counts of platelets, neutrophils, leukocytes, reticulocytes, and lymphocytes than those that died after day 12 (P<0.001 to P<0.05). When compared by treatment group, animals treated with 100 ng/Kg rHuIL-12 had significantly higher counts of neutrophils, leukocytes, and lymphocytes than did those treated with vehicle in both survivors and decedent groups (P<0.001 for all three cell types). In addition, animals treated with 100 ng/Kg rHuIL-12 had a numerically higher platelet and reticulocyte counts. These findings show that rHuIL-12-induced increase in blood cell counts around nadir play a key role in promoting survival following radiation exposure.

### Example 19

### Clinical and Physical Characteristics of Irradiated Monkeys Following rHuIL-12 Administration

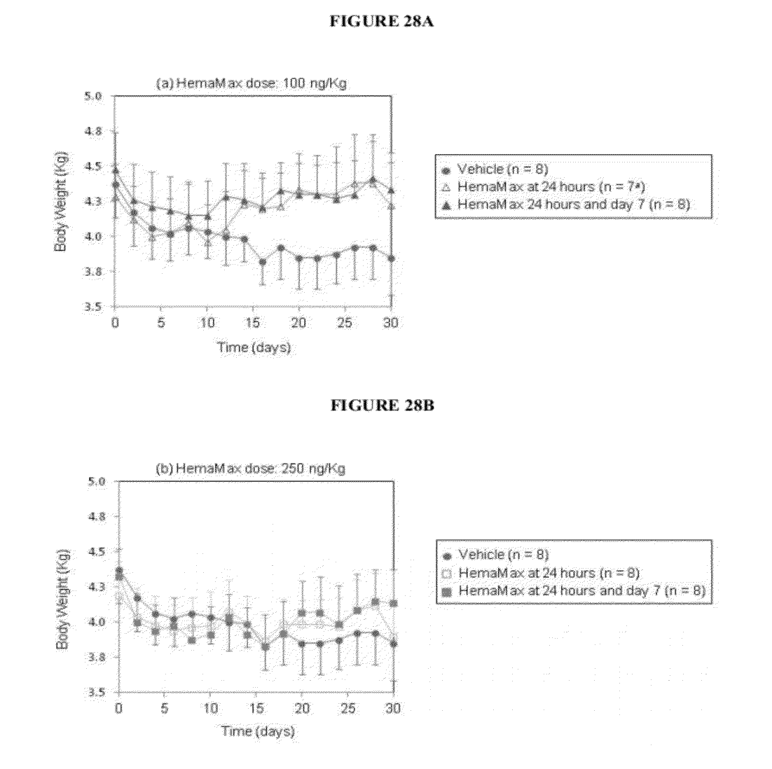

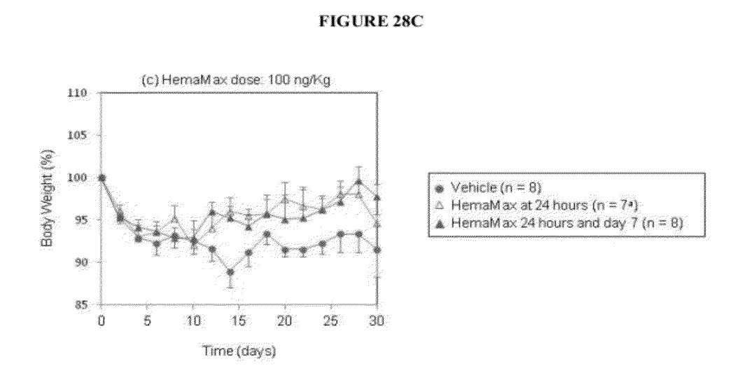

Non-human primates (NHP) treated as described in Example 17 were observed for their clinical and physical characteristics. Animals receiving rHuIL-12 at the dose of 100 ng/Kg (once or twice) had consistently higher mean body weights than did those in the vehicle group from days 14 to day 30 (FIG. 28A). Animals treated with rHuIL-12 at the dose of 100 ng/Kg (once or twice) or 250 ng/Kg (once) had less weight loss than did animals treated with vehicle from days 14 to 30 (FIGS. 28C-D).

Although the between group differences in body weight or weight loss were not statistically significant, when the analysis of body weight loss was limited to day 12—the approximate day for blood cell nadir and the day after which animals began to die (see FIG. 26A)—the pooled rHuIL-12-treated animals had significantly less body weight loss than those treated with vehicle (95.3±0.8% versus 91.6±1.5%, respectively; P=0.04). Logistic regression demonstrated that weight loss after day 12 was a strong predictor of survival (P<0.001). Other clinical signs (appetite, physical activity, diarrhea, and feces color) were not significantly different from the vehicle group, although appetite and physical activity improved in rHuIL-12 treated animals, and the incidence of diarrhea and black or red feces declined in the 250 ng/Kg twice dosing regimen group. However, above-mentioned clinical signs did predict mortality after day 12 by logistic regression (P=0.002 for decreased appetite, P<0.001 for decreased physical activity, P=0.04 for incidence of diarrhea, and P=0.008 for incidence of red or black feces). Clinical signs of severe deterioration and stress, including chronic anorexia, sunken eyes, dehydration, hunched and/or crouching posture and weakness, started approximately at day 14 with no remarkable between-group differences in the incidence or onset. All adverse clinical signs were consistent with acute radiation syndrome following exposure to radiation.

Gross pathology along with organ and hemoculture bacteriology evaluation was conducted for all animals, which died or were euthanized before the end of study. There were no rHuIL-12—related macroscopic lesions. The incidence of hemorrhage was 12.5% (1/8 animals) in the pooled animals treated with 100 ng/Kg or 250 ng/Kg of rHuIL-12 compared to 50% (2/4) in the vehicle animals. In the vehicle group, all of the decedent animals (4/8 animals) were found dead while only 1 animal in the rHuIL-12 groups was found dead and 8 animals were humanely euthanized before the end of study. A diagnosis of septicemia was confirmed by isolation of the same bacterial strain in at least 2 organs of all 13 animals.

In the vehicle group, 75% (3/4) found dead animals presented a combination of bacteria most likely from the intestinal and cutaneous flora and 25% (1/4) presented organ infections with only bacteria most likely from the cutaneous bacterial flora. In the various rHuIL-12-treated groups, 8 out of 9 animals (89%) presented a combination of bacteria from the intestinal and cutaneous flora, including 2 which also presented organ infections with bacteria most likely from the environment. The other animal (1/9) presented organ infections with only bacteria most likely from the cutaneous flora. These results suggest that opportunistic infections were present in all animals that died preterminally in this animal model of acute radiation syndrome.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the invention claimed. Thus, it should be understood that although the present invention has been specifically disclosed by preferred embodiments and optional features, modification and variation of the invention embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein.

Other embodiments are within the following claims. In addition, where features or aspects of the methods are described in terms of Markush groups, those skilled in the art will recognize that the invention is also thereby described in terms of any individual member or subgroup of members of the Markush group.

I claim:

1. A method for increasing the probability of survival in a subject, the method comprising administering at least one therapeutically effective dose of IL-12 to the subject following an acute exposure to non-therapeutic whole body ionizing radiation, wherein the subject's probability of survival is increased due to at least one or two effects selected from the group consisting of:
(a) stimulation of innate immunity effects;
(b) protection of the gastrointestinal tract;
(c), stimulation of hematopoiesis, and
(d) stimulation of antioxidant and anti-apoptotic effects.

2. The method of claim 1, wherein the subject is human.

3. The method of claim 1, wherein the stimulation of antioxidant and anti-apoptotic effects results from release of erythropoietin (EPO).

4. The method of claim 1, wherein the stimulation of innate immunity effects results from release of interferon-gamma.

5. The method of claim 1, wherein the probability of survival is further increased by the release of endogenous IL-12.

6. The method of claim 1, wherein the subject has a decreased probability of infection, a decrease in tissue damage, or a combination thereof, as compared to a subject exposed to the same level of non-therapeutic whole body ionizing radiation and who has not been given a dose of IL-12.

7. The method of claim 1, wherein IL-12 directly stimulates the innate immunity effects by binding to the IL-12 receptor on natural killer cells, macrophages, dendritic cells, or any combination thereof.

8. The method of claim 2, wherein the dose of IL-12 is less than about 300 ng/kg or less than about 100 ng/kg.

9. The method of claim 1, wherein the acute exposure to whole body ionizing radiation is the result of a nuclear event.

10. The method of claim 1, wherein the acute exposure is at least about 1.0 Gy.

11. The method of claim 1, wherein the acute exposure is less than about 3.0 Gy.

12. The method of claim 1, wherein the IL-12 is administered:
(a) between a range of about 6 hours to about 120 hours after the acute radiation exposure;
(b) between a range of about 6 hours to about 72 hours after the acute radiation exposure; or
(c) at about 6 hours, about 12 hours or about 24 hours after the acute radiation exposure.

13. The method of claim 1, wherein IL-12 is administered subcutaneously or intramuscularly.

14. The method of claim 1, wherein supportive care is given to the subject simultaneously or following the administration of IL-12.

15. The method of claim 14, wherein supportive care comprises one or more of the following:
(a) administration of one or more antibiotics;
(b) administration of one or more hematopoietic growth factors; and
(c) administration of a blood transfusion.

16. The method of claim 15, wherein the hematopoietic growth factors are selected from the group consisting of G-CSF, GM-CSF and EPO.

17. The method of claim 1, wherein the subject has a decreased need for a platelet transfusion, a decreased probability of sepsis, a decreased probability of hemorrhage, or any combination thereof, as compared to a subject exposed to the same level of non-therapeutic whole body ionizing radiation and who has not been given a dose of IL-12.

18. A method for resorting hematopoiesis in a subject, the method comprising administering at least one therapeutically effective dose of IL-12 to the subject following an acute exposure to non-therapeutic whole body ionizing radiation, wherein the dose is administered at least 24 hours after radiation exposure and hematopoiesis is restored via activation of the IL-12 receptor on hematopoietic cells in the bone marrow.

19. The method of claim 18, wherein the hematopoietic cells comprise niche cells and stem cells.

20. The method of claim 19, wherein the niche cells comprise osteoblasts.

21. The method of claim 18, where the hematopoiesis is restored following activation of the IL-12 receptor on megakaryocytes.

22. The method of claim 21, wherein the megakaryocytes are immature.

23. The method of claim 18, wherein hematopoiesis is restored following activation of the IL-12 receptor on osteoblastic cells in the bone marrow, megakaryocyte cells in the bone marrow, hematopoietic stem cells in the bone marrow, or a combination thereof.

24. The method of claim of claim 18, wherein the therapeutically effective dose of IL-12 is administered between about 48 and about 120 hours following radiation exposure.

25. The method of claim 18, wherein the dose of IL-12 is less than about 100 ng/kg or less than about 300 ng/kg.
